(19) United States
(12) Patent Application Publication
(10) Pub. No.: US 2020/0163850 A1
(43) Pub. Date: May 28, 2020

(54) USE OF POLYHYDROXYAROMATIC COMPOUNDS FOR THE TREATMENT OF FIBROUS AMINO ACID BASED SUBSTRATES

(71) Applicant: Momentive Performance Materials GmbH, Leverkusen (DE)

(72) Inventors: Roland Wagner, Bonn (DE); Anne Dussaud, Tarrytown, NY (US); Nicholas Stasiak, Putnam Valley, NY (US); Kunshan Sun, Millwood, NY (US); Katharina Streicher, Leverkusen (DE); Shrutisagar Haveli, Bibwewadi Pune (IN)

(21) Appl. No.: 16/208,787

(22) Filed: Dec. 4, 2018

(30) Foreign Application Priority Data
Nov. 24, 2018 (IN) 201821044328

(51) Int. Cl. A61K 8/37; A61K 8/65; A61Q 5/02; A61Q 5/12; A61Q 5/06; C07C 69/84

(52) U.S. Cl. CPC A61K 8/375; A61K 8/65; A61Q 5/02; A61K 2800/524; A61Q 5/065; C07C 69/84; A61Q 5/12

(57) ABSTRACT

This invention relates to the use of polyhydroxyaromatic compounds for the treatment of fibrous amino acid based substrates, preferred hair, new polyhydroxyaromatic compounds, aqueous compositions comprising the polyhydroxyaromatic compounds, cosmetic compositions comprising the polyhydroxyaromatic compounds, in particular hair care compositions, and a process for the treatment of hair comprising the use of said cosmetic compositions.

### SUMMARY

This invention relates to the use of polyhydroxyaromatic compounds for the treatment of fibrous amino acid based substrates, preferred hair, new polyhydroxyaromatic compounds, aqueous compositions comprising the polyhydroxyaromatic compounds, cosmetic compositions comprising the polyhydroxyaromatic compounds, in particular hair care compositions, and a process for the treatment of hair comprising the use of said cosmetic compositions.

### TECHNICAL FIELD

This invention relates to the use of polyhydroxyaromatic compounds and aqueous compositions comprising the same for the treatment of fibrous amino acid based substrates, preferred hair, in particular, human and animal hair, more specifically human hair. Of further interest are also long term storage stable aqueous compositions, which deliver and attach the polyhydroxyaromatic compounds to human hair for permanent shaping and coloration purposes.

### BACKGROUND OF THE INVENTION

Hair generally can be straight, wavy, curly, kinky or twisted. A human hair includes three main morphological components, the cuticle (a thin, outer-most shell of several concentric layers), the cortex (the main body of the hair), and, in case of higher diameter hair, the medulla (a thin, central core). The cuticle and cortex provide the hair strand's mechanical properties, that is, its tendency to have a wave, curl, or kink. A straight hair strand can resemble a rod with a circular cross-section, a wavy hair strand can appear compressed into an oval cross-section, a curly strand can appear further compressed into an elongated ellipse cross-section, and a kinky hair strand cross-section can be flatter still. The primary component of hair is the cross-linked, α-helix protein keratin. Keratins are intermediate filament proteins found specifically in epithelial cells, e.g. human skin and hair, wool, feathers, and nails. The α-helical type I and II keratin intermediate filament proteins (KIFs) with molecular weights around 45-60 kDa are embedded in an amorphous matrix of keratin-associated proteins (KAPs) with molecular weights between 20 to 30 kDa (M. A. Rogers, L. Langbein, S. Praetzel-Wunder, H. Winter, J. Schweizer, J. Int Rev Cytol. 2006; 251:209-6); both intra- and intermolecular disulfide bonds provided by cystines contribute to the cytoskeletal protein network maintaining the cellular scaffolding. In addition to the disulfide cross-links ionic bonding or salt bridges which pair various amino acids found in the hair proteins contribute to the hair strand's outward shape.

It is well known in the art that hair, can be treated with functionalized silicones which deliver one or more cosmetic benefits, such as conditioning, color retention, shine and UV protection. Typically, these silicones are physically deposited on the fiber surface (cuticle) and therefore responsible for the outward appearance of the hair. They can be removed partially or completely by repeated washing processes. While the deposited silicones considerably improve the surface properties of hair, i.e. smoothness and friction, they do not substantially impact the mechanical properties and the shape of the fibers.

People with naturally wavy, curly, or kinky hair may desire to reduce fizz and to get more control and a smoother appearance of their hairs. Several hair treatments including straightening methods are available, but these often involve the use of harsh and regulated substances.

Frequently used straightening preparations are based of sodium or potassium hydroxide blended with starch which are highly irritating to the scalp. Less irritating formulations are based on guanidine hydroxide or certain sulfites. Recent formulations are based on thio glycolic acid salts. The underlying principle is that under alkaline conditions disulfide bonds within the hair proteins undergo a reductive cleavage. The disulfide bonds are reduced to sulfhydryls and after shaping the desired hair configuration re-established by oxidation.

The different processes for straightening hairs have the tendency to weaken the strength of the hair. Therefore it is desirable to find compositions which enable straightening and recover strength and elasticity of the hair. Traditionally, aldehyde based formulations for a permanent hair shaping were developed. Most frequently formaldehyde is used for this so called Brazilian keratin shaping method (US 2012-0031420). The underlying principle is the crosslinking reaction between formaldehyde and keratin based amino and amido groups (H. Puchtler, Histochemistry, 82(1985), pp. 201-204) or between formaldehyde and SH groups (US 2009-0211593) after straightening. Dialdehydes, i.e. glyoxal, were proposed to replace formaldehyde (US 2009-0165812). The inventors were looking for alternative, non-irritating compounds which can replace the critical formaldehyde treatment.

Polyhydroxyaromatic compounds containing glycerol derivatives are known. The synthesis of mono-, di and trigalloyl glycerol is described in U.S. Pat. No. 1,927,339. Starting point are the corresponding mono-, di- and trichloro derivatives of glycerol. Monogalloyl glycerol is known as an antioxidant (Z. Song, Z. Xiao, Linchan Huaxue Yu Gongye (1988), 8(3), 9-18). Monogalloyl di-oleyl glycerol causes a weight loss in rats (T. Nagao, T. Sayuri, T. Tatsuya, M. Yukari, S. Hideaki Journal of Oleo Science (2011), 60(9), 457-62). Polygalloyl derivatives, i.e. hexagalloyl sorbitol, pentagalloyl glucose and trigalloyl glycerol were proposed in tanning compositions (WO 9218457). Galloyl diglucoside is a component in hair care formulations reducing hair damage (JP 2004210724). Naturally derived tannins, i.e tannic acid as a complex polygalloyl ester of glucose, are used in in hair dyeing formulations (U.S. Pat. No. 4,981,485).

WO2016046178 discloses polyhydroxyaromatic silicones for the treatment of hair.

None of the above prior art disclosures describes a straight forward methodology towards well defined galloyl derivatives of hydrocarbons where the backbone chain can be flexibly adjusted within a wide range. Further, none of the prior art describes methodologies yielding long term stable, easy to prepare and easy to use compositions containing these well-defined galloyl hydrocarbon derivatives and which are in addition robust towards the presence of other performance ingredients and which provide a hair strengthening benefit and a hair coloration benefit without the usage of strongly irritating auxiliaries.

### SUMMARY OF THE INVENTION

In accordance with the present invention there is provided the use of compound of the formula:

R²(—F)₂₋₁₈

wherein

R² is selected from divalent to octadecavalent, optionally substituted, preferably aliphatic hydrocarbon radicals which have up to 100 carbon atoms, and may contain optionally one or more groups selected from —O—, —NH—, —C(O)—, —C(S)—, tertiary amino groups

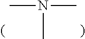

and quaternary ammonium groups and

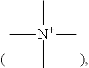

F is selected from selected from:

—O—C(O)—R³—R⁴, and

—NR¹—C(O)—R³—R⁴,

(preferably F is selected from selected from —O—C(O)—R³—R⁴),
the groups F bind to a carbon atom of R²,
wherein
R¹ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

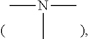

and quaternary ammonium groups

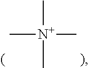

R³ is selected from a single bond or optionally substituted straight-chain, cyclic or branched, saturated, unsaturated or aromatic hydrocarbon radicals which have up to 100 carbon atoms, which optionally contain one or more groups selected from —O—, —NH—, —C(O)—, —C(S)—, tertiary amino groups

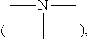

and quaternary ammonium groups

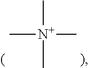

preferably R³ is a single bond, or an ethenylene group, and
R⁴ is selected from the group consisting of di- and trihydroxy-substituted aromatic groups,
wherein the compound excludes tannins,
for the treatment of fibrous amino acid based substrates, preferred hair. Said use involves also a method for the treatment of hair, which comprises the application of said compound on said hair, in particular method for the cosmetic treatment of hair.

The claimed use or method does not include the use of tannins as the compounds, although the combined use of said compounds together with tannins is covered by the scope of the invention. According to Karamali Khanbabaee and Teunis van Ree, Nat. Prod. Rep., 2001, 18, 641-649 “Tannins: Classification and Definition” tannins are “polyphenolic secondary metabolites of higher plants, and are either galloyl esters and their derivatives, in which galloyl moieties or their derivatives are attached to a variety of polyol-, catechin- and triterpenoid cores (gallotannins, ellagitannins and complex tannins), or they are oligomeric and polymeric proanthocyanidins that can possess different interflavanyl coupling and substitution patterns (condensed tannins)”. Such tannins are excluded from the scope of the compounds of formula R²(—F)₂₋₁₈.

The groups F are:

—O—C(O)—R³—R⁴, and

—NR¹—C(O)—R³—R⁴,

wherein the bond marked with the asterisk bind to a carbon atom of R², schematically in the manner:

C*—O—C(O)—R³—R⁴, and

C*—NR¹—C(O)—R³—R⁴.

The preferred group F is —O—C(O)—R³—R⁴.

In a preferred embodiment of the invention R⁴, the di- and trihydroxy-substituted aromatic groups, is a group of the formula:

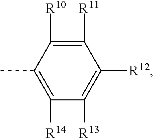

wherein R¹⁰, R¹¹, R¹², R¹³, R¹⁴ are independently selected from R¹, preferably R¹ is hydrogen, or hydroxyl, with the proviso that 2 to 3 groups of R¹⁰ to R¹⁴ are hydroxyl, and wherein the dotted line represents the bond to R³, or is R³ in case R³ is a single bond, or R⁴ is selected from the group consisting of di- and trihydroxy-substituted naphthyl groups.

Particularly preferred groups R⁴ are selected from the group consisting of dihydroxyphenyl groups, such as 2,3-dihydroxyphenyl, 2,4-dihydroxyphenyl, 2,5-dihydroxyphenyl, 2,6-dihydroxyphenyl, 3,4-dihydroxyphenyl, and 3,5-dihydroxyphenyl, trihydroxyphenyl groups, such as 2,3,4-trihydroxyphenyl, 2,3,5-trihydroxyphenyl, 2,3,6-trihydroxyphenyl, 2,4,5-trihydroxyphenyl, 2,4,6-trihydroxyphenyl, and 3,4,5-trihydroxyphenyl, dihydroxynaphthyl groups, such as 6,7-dihydroxynaphthyl, 5,6-dihydroxynaphthyl, 1,7-dihydroxynaphthyl, 3,7-dihydroxynaphthyl, 1,3-dihydroxynaphthyl, and 1,4-dihydroxynaphthyl, and trihydroxynaphthyl groups, such as 4,6,7-trihydroxynaphthyl, and 1,5,6-trihydroxynaphthyl. Most preferred is 3,4,5-trihydroxyphenyl.

In a preferred embodiment of the invention R⁴ is derived from polyhydroxy aromatic carboxylic acids such as poly (i.e. di- and trihydroxy)hydroxy benzoic acids, such as dihydroxy benzoic acids, e.g. 2,3-dihydroxy benzoic acid, 2,4-dihydroxy benzoic acid, 2,5-dihydroxy benzoic acid, 2,6-dihydroxy benzoic acid, 3,4-dihydroxy benzoic acid, and 3,5-dihydroxy benzoic acid, or trihydroxy benzoic acids, e.g. 2,3,4-trihydroxy benzoic acid, 2,3,5-trihydroxy benzoic acid, 2,3,6-trihydroxy benzoic acid, 2,4,5-trihydroxy benzoic acid, 2,4,6-trihydroxy benzoic acid, and 3,4,5-trihydroxy benzoic acid, polyhydroxy cinnamic acids, such as dihydroxy cinnamic acids, e.g. 3,4-dihydroxy cinnamic acid, or trihydroxy cinnamic acids, e.g. 3,4,5-dihydroxy cinnamic acid, polyhydroxynaphthalene carboxylic acids, such as dihydroxynaphthalene carboxylic acids, e.g. 6,7-dihydroxynaphthalene-2-carboxylic acid, 5,6-dihydroxynaphthalene-2-carboxylic acid, 1,7-dihydroxynaphthalene-2-carboxylic acid, 3,7-dihydroxynaphthalene-2-carboxylic acid, 1,3-dihydroxynaphthalene-2-carboxylic acid, and 1,4-dihydroxynaphthalene-2-carboxylic acid, or trihydroxynaphthalene carboxylic acids, e.g. 4,6,7-trihydroxynaphthalene-2-carboxylic acid, and 1,5,6-trihydroxynaphthalene-2-carboxylic acid. Most preferred is 3,4,5-trihydroxy benzoic acid (gallic acid).

Here and in the context of the present invention “derived from” shall mean in particular, residues that are formally formed from compounds by reacting those compounds to arrive at the compounds of the invention. For example, considering the embodiment where R⁴ is derived from polyhydroxy aromatic carboxylic acids, as mentioned above, in such case R⁴ derived from polyhydroxy aromatic carboxylic acids would result e.g. from the reaction of a polyol or a polyepoxide of formulas:

R²(-A)_{c}

wherein A is OH and/or an epoxy group, preferred epoxy group:

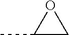

and wherein c=2-18, preferably c=2 to 10, more preferably c=2 to 8, more preferably c =2 to 4, with the polyhydroxy aromatic carboxylic acids H—O—C(O)—R³—R⁴, to form e.g.

R²(—OH)ₐ(—O—C(O)—R³—R⁴)_{b}

with b≥2 and a+b=2 to 18, preferably b=2 to 6, more preferably b=2 to 4, more preferably b=2, and a+b=preferably 2 to 8, more preferably a+b=2 to 6,
and accordingly R⁴ derived from polyhydroxy aromatic carboxylic acids would be di- or trihydroxy-substituted aromatic group, and R³ a single bond.

In a preferred embodiment of the invention the optional substituents of the groups R¹, R² and R³ are selected from the groups consisting of hydroxyl, amino and halogen, preferably hydroxyl and amino, and the number of the substituents may be up to 5, preferably 1 to 4.

In a preferred embodiment of the invention R¹ is selected from the group consisting of hydrogen, n-, iso-, or tert.-C₁-C₂₂-alkyl, C₂-C₂₂-alkoxyalkyl, C₅-C₃₀-cycloalkyl, C₆-C₃₀-aryl, C₆-C₃₀-aryl(C₁-C₆)alkyl, C₆-C₃₀-alkylaryl, C₂-C₂₂-alkenyl, C₂-C₂₂-alkenyloxyalkyl, which optionally can be each substituted by hydroxyl and halogen, and which optionally can contain one or more ether groups (—O—), preferably hydrogen or n-, iso-, or tert.-C₁-C₂₂-alkyl. Most preferred R¹ is hydrogen.

In a preferred embodiment of the invention R² is selected from divalent to decavalent, more preferred divalent to hexavalent, even more preferred divalent, preferably aliphatic, hydrocarbon radicals which have 2 to 30 carbon atoms, more preferred 2 to 20 carbon atoms, even more preferred 2 to 15 carbon atoms and may contain optionally one or more groups selected from —O—, —NH—, —C(O)—, —C(S)—, and wherein R² may optionally be substituted by one or more hydroxyl groups.

In a preferred embodiment of the invention R² has a valency of 2 to 18, preferably 2 to 12, more preferably 2 to 10, and still more preferably 2 to 8, such as 2 to 6, specifically 2 or 3 or 4.

In a preferred embodiment of the invention R² is selected from R²′ being selected from the group consisting of:
divalent to octadecavalent, preferably divalent to hexavalent hydrocarbyl groups, more preferably divalent hydrocarbyl groups, derived from aliphatic polyols having more than three carbon atoms such as alkane di-, tri- and tetraols, e.g. 1,6 hexandiol, trimethylolpropane, and pentaerythritol,
divalent to octadecavalent, preferably divalent to hexavalent hydrocarbyl groups, more preferably divalent hydrocarbyl groups, comprising at least one ether group, and optionally having one or more hydroxyl substituents, derived from polyalkyleneoxides, such as ethylene oxide-, propylene oxide- and/or butylene oxide-based polyethers, e.g. derived from polyethylene glycols, like diethylene glycol, triethylene glycol, tetraethylene glycol, and pentaethylene glycol etc., or derived from polypropylene glycols, like dipropylene glycol (e.g, derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, etc., derived from mixed ethylene oxide and butylene oxide based copolyethers, derived from mixed propylene oxide and butylene oxide based copolyethers, and derived from mixed ethylene oxide and propylene oxide and butylene oxide based copolyethers,
divalent to octadecavalent, preferably divalent to hexavalent hydrocarbyl groups, more preferably divalent hydrocarbyl groups, optionally comprising at least one ether group, and optionally having one or more hydroxyl substituents, derived from oligoglycerols, such as diglycerol, triglycerol, tetraglycerol, pentaglycerol, hexaglycerol, and the statistically distributed oligomeric condensation products of glycerol, such as
divalent to octadecavalent, preferably divalent to hexavalent hydrocarbyl groups, more preferably divalent hydrocarbyl groups, derived from compounds comprising at least one glycidoxy group, such as diglycidyl ether, glycerol diglycidyl ether, and glycerol triglycidylether,
divalent to octadecavalent, preferably divalent to hexavalent hydrocarbyl groups, more preferably divalent hydrocarbyl groups, comprising at least one ether group, and optionally having one or more hydroxyl substituents, derived from polyol alkylene oxide addition products, e.g. the addition products of ethylene oxide and/or propylene oxide to polyols such as ethylene glycol, 1,2 propylene glycol, glycerol, trimethylolpropane, pentaerythritol, sorbitol and sucrose,
divalent to octadecavalent, preferably divalent to hexavalent hydrocarbyl groups, more preferably divalent hydrocarbyl groups, comprising at least one ether group, and optionally having one or more hydroxyl and/or amino substituents, derived from polyamine alkylene oxide addition products, e.g. the addition products of ethylene oxide and/or propylene oxide to ethylenediamine, diethylene triamine, or derived from alkylene oxide addition products of ethanol amine,
divalent to octadecavalent, preferably divalent to hexavalent hydrocarbyl groups, more preferably divalent hydrocarbyl groups, comprising at least one ester group, and having one or more hydroxyl substituents, e.g. derived from polyesters, preferably derived from the condensation of di- to hexavalent carboxylic acids, e.g. maleic acid, succinic acid, adipic acid, sebacic acid, itaconic acid, tartaric acid, trimellitic acid with alkylene oxides, such as ethylene oxide, propylene oxide, butylene oxide, and compounds comprising at least one glycidoxy group, such as glycidol, diglycidyl ether, glycerol diglycidyl ether, glycerol triglycidyl ether, in particular the condensation products of succinic acid, maleic acid and tartaric acid to glycerol diglycidyl ether,
further R²′ presents preferably polyalkylene oxy groups, preferably of the general formula:

—[CH₂CH₂O]_{q1}—[CH₂CH(CH₃)O]ᵣ₁—[CH₂CH(C₂H₅)O]ₛ₁—{[CH₂CH₂]_{q2}—[CH₂CH(CH₃)]ᵣ₂—[CH₂CH(C₂H₅)]ₛ₂}—

with
q1=0 to 49, preferred 0 to 10, more preferred 1 to 10, even more preferred 1 to 5,
r1=0 to 32, preferred 0 to 10, more preferred 1 to 10, even more preferred 1 to 5,
s1=0 to 24, preferred 0 to 10, more preferred 1 to 10, even more preferred 1 to 5,
q2=0 or 1,
r2=0 or 1,
s2=0 or 1, and
Σ(q2+r2+s2)=1,
with the proviso that the sum of the carbon atoms in such polyalkylene oxide groups is 2 to 100, preferred 2 to 50, more preferred 2 to 30, even more preferred 2 to 20, specific 2 to 15,
divalent hydrocarbyl groups, derived from oligoglycerols of the general formula:

—[CH₂CH(R⁷)CH₂O]ₜ₁—[CH₂CH(R⁷)CH₂)]ₜ₂—

with
t1=0 to 32, preferred 0 to 10, more preferred 1 to 10, even more preferred 1 to 5, specifically 1 and 2,
t2=1,
R⁷═OH or F, wherein F is as defined above, preferably —O—C(O)—R³—C(O)OH,
with the proviso that the sum of the carbon atoms is 2 to 100, preferred 2 to 50, more preferred 2 to 30, even more preferred 2 to 20, specific 2 to 15, and
divalent hydrocarbyl groups, comprising at least one ester group of the general formulae:

—[CH₂CH₂O]_{q1}—R⁸—[CH₂CH₂O]_{q1}—[CH₂CH₂]_{q2}—

with q1 can be the same or different and are as defined above and q2=1
and

—[CH₂CH(R⁷)CH₂O]ₜ₁—R⁸—[CH₂CH(R⁷)CH₂O]ₜ₁—[CH₂CH(R⁷)CH₂)]ₜ₂—

with t1, t2 and R⁷ as defined above and
R⁸ being selected from —C(O)C(O)O—, —C(O)(CH₂)₁₋₈C(O)O—, such as being derived from succinic acid, adipic acid, sebacic acid, or —C(O)(C₆H₄)C(O)O—, i.e. derived from phthalic and terephthalic acid, —C(O)CH═CHC(O)O—, —C(O)C(═CH₂)—CH₂C(O)O—, —C(O)CH(OH)CH(OH)C(0)O—,
with the proviso that the sum of the carbon atoms in R²′ is 2 to 100, preferred 2 to 50, more preferred 2 to 30, even more preferred 2 to 20, specifically 2 to 15.

In a preferred embodiment of the invention R² (or R²′, or R²″ as mentioned above or below) contains one or more groups —O—, such as one to five. These groups —O— are preferably ether groups, but can also form an ester group together with a carbonyl group. Preferably these groups R² (or R²′, or R²″ as mentioned above or below) are substituted by one or more hydroxyl groups.

In a preferred embodiment of the invention the compound used according to the invention has the formula:

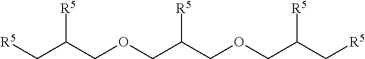

wherein R⁵ is selected from the group consisting of hydroxy or F, wherein F is as defined above, with the proviso that at least two of R⁵ are F.

In a preferred embodiment of the invention the compound used according to the invention has the formula has the formula:

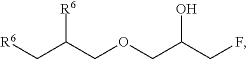

wherein one of R⁶ is hydroxy and one of R⁶ is a group of the formula

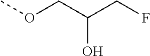

wherein F is as defined above and the dotted line is the bond to the carbon atom. Said compound is preferably a mixture of the following two isomers:

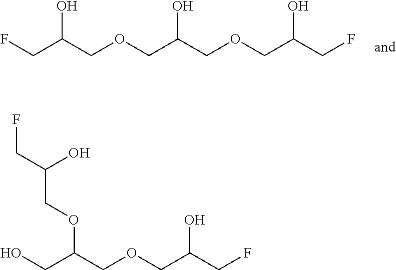

wherein F is as defined above.

In a preferred embodiment the compound used according to the invention has the formula:

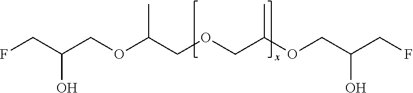

wherein x is from 1 to 10, preferably 1 to 5, and F is as defined above.

In a preferred embodiment R³ is selected from a single bond and straight-chain, cyclic or branched, saturated, unsaturated or aromatic hydrocarbon radicals which have up to 18, preferably up to 12, more preferably up to 10 carbon atoms, and which optionally contain one or more groups selected from —O—, —NH—, —C(O)—, and wherein R³ is optionally substituted by one or more groups selected from hydroxyl groups, amino groups, and carboxy groups, preferably R³ is single bond or ethenylene.

In a preferred embodiment of the invention R² is an aliphatic hydrocarbyl group which is not derived from a hexose. Hexoses are monosaccharides with six carbon atoms, having the chemical formula C₆H₁₂O₆ and include aldohexoses and their cyclic hemiacetals, and ketohexoses. In particular, they include glucose, which is thus preferably excluded.

In a further preferred embodiment of the invention R² is not derived from a carbohydrate (saccharide), such as monosaccharides, disaccharides, oligosaccharides, and polysaccharides, or a sugar alcohol. A carbohydrate or saccharide is a biomolecule consisting of carbon (C), hydrogen (H) and oxygen (O) atoms, usually with a hydrogen-oxygen atom ratio of 2:1 (as in water) and thus with the empirical formula Cₘ(H₂O)ₙ (where m may be different from n). They also include deoxyribose, and structurally include in particular aldoses and ketoses, residues derived from which are thus preferably excluded from R².

Sugar alcohols (also called polyhydric alcohols, polyalcohols, alditols or glycitols) are organic compounds, typically derived from sugars, that comprise a class of polyols, and commonly include e.g. ethylene glycol, glycerol, erythritol, threitol, arabitol, xylitol, ribitol, mannitol, sorbitol, galactitol, fucitol, iditol, inositol etc. (see e.g. https://en.wikipedia.org/wiki/Sugar_alcohol). In particular, R² derived from ethylene glycol and glycerol are preferably excluded.

In a further preferred embodiment of the invention R² is not derived from carbohydrate- or sugar alcohol-esters of gallic acid.

The present invention also relates to compounds of the formula:

R²″(—F)₂₋₁₈

wherein R²″ is selected from divalent to octadecavalent, optionally substituted, aliphatic hydrocarbon radicals which have up to 100 carbon atoms, and may contain optionally one or more groups selected from —O—, —NH—, —C(O)—, —C(S)—, tertiary amino groups

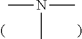

and quaternary ammonium groups

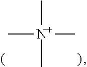

and
F is selected from selected from:

—O—C(O)R³—R⁴, and

—NR¹—C(O)R³—R⁴,

the groups F bind to a carbon atom of R²″, wherein R²″ is not derived from a carbohydrate (saccharide) or a sugar alcohol (regarding the definition of carbohydrate (saccharide) or a sugar alcohol it is referred to the above explanations), and
wherein
R¹ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

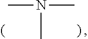

and quaternary ammonium groups

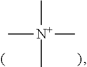

R³ is selected from a single bond or optionally substituted straight-chain, cyclic or branched, saturated, unsaturated or aromatic hydrocarbon radicals which have up to 100 carbon atoms, which optionally contain one or more groups selected from —O—, —NH—, —C(O)—, —C(S)—, tertiary amino groups

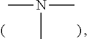

and quaternary ammonium groups

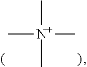

preferably R³ is a single bond, or an ethenylene group, and
R⁴ is selected from the group consisting of di- and trihydroxy-substituted aromatic groups.

These compounds according to the invention preferably exclude tannins as defined above.

The present invention further relates to compounds of the formula:

R²′('F)₂₋₁₈

wherein R²′ and F are each as defined above.

The present invention further relates to compounds of the formula:

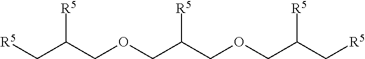

wherein R⁵ is selected from the group consisting of hydroxy or F, wherein F is as defined above, with the proviso that at least two of R⁵ are F.

The present invention further relates to compounds of the formula:

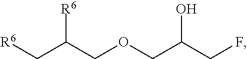

wherein one of R⁶ is hydroxy and one of R⁶ is a group of the formula

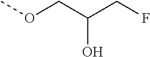

wherein F is as defined above and the dotted line is the bond to the carbon atom.

The present invention further relates to a mixture of the following two compounds:

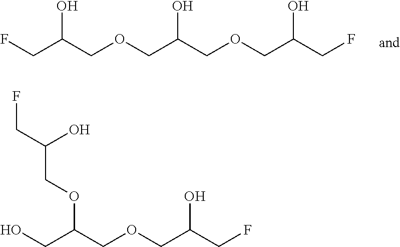

wherein F is as defined above.

Examples for precursors for glycerol moieties containing compounds according to the invention are the glycidyl functional Denacol types EX-313, EX-314, EX-421, EX 512, EX 521 (Nagase).

Examples for precursors for trimethylolpropane, pentaerythrol and neopentyl glycol moieties containing compounds according to the invention are the glycidyl functional Denacol types EX-321, EX-411 and EX-211 (Nagase).

In another preferred embodiment of the present invention further relates to compounds of the formula:

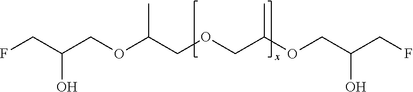

wherein x is from 1 to 10, preferably 1 to 5, and F is as defined above.

Examples for precursors for propylene glycol moieties containing compounds according to the invention are the glycidyl functional Denacol types EX-920, EX-921 (Nagase).

Examples for precursors for ethylene glycol moieties containing compounds according to the invention are the glycidyl functional Denacol types EX-821, EX-830, EX-832, EX-841, EX-850, EX-851, EX-861 (Nagase).

Another embodiment of the present invention relates to an aqueous composition comprising at least one compound according to the invention as described above. In a preferred embodiment of the aqueous composition according to the invention it comprises optionally at least one further component selected from the group consisting of surfactants, and metal salts, preferably Zn²⁺, Fe²⁺ and/or Fe³⁺ salts.

In a preferred embodiment of the aqueous composition according to the invention the weight ratio of the optional surfactant and/or metal salt to said compound according to the invention is at least 0.06, more preferred 0.06 to 5, more preferred 0.06 to 3, even more preferred 0.1 to 3, specifically 0.1 to 1.

The aqueous composition according to the invention comprises preferably 0.05 to 30%, more preferred 0.5 to 30%, more preferred 1 to 30%, even more preferred 1 to 20%, specifically 1 to 10% of said inventive compound, wherein each percentage is per weight based on the total weight of the aqueous composition.

The amount of the surfactant present in the aqueous composition is preferably from about 0.05% to about 15%, more preferably from about 0.05% to about 5%, still more preferably from about 0.1% to about 5%, specifically from 0.1 to 3% by weight of the aqueous composition.

Preferably the surfactant is selected from cationic, nonionic, betaine and anionic surfactants, preferably having a HLB value ranging from 1 to 20, preferred 7 to 20, more preferred 8 to 20. A preferred surfactant is selected from hydrocarbon-based or silicone-based emulsifiers. The cationic surfactant is preferably selected from primary, secondary, or tertiary amine compounds having up to 50 carbon atoms and salts thereof, amido amine compounds having up to 50 carbon atoms and salts thereof, such as behenamidopropyl dimethylamine and quaternary ammonium compounds, having up to 50 carbon atoms, and preferably with up to 20 carbon atoms in the alkyl groups thereof, such as tetraalkyl ammonium compounds, e.g. hexadecyl-trimethylammonium salts, dimethyldioctadecylammonium salts, distearyldimethylammonium salts, cetrimonium salts, cetylpyridinium salts, alkylbenzyldimethylammonium salts such as benzalkonium salts, benzethonium salts, ester quats having at least one quaternary ammonium group and at least one ester group.

Further preferred examples for cationic emulsifiers are quaternary ammonium groups or amino groups containing linear or branched C8 to C50, preferred C8 to 40, more preferred C8 to C30 alkyl, fatty alcohol and fatty acid based emulsifiers, i.e. fatty acid based ester quats containing one or two fatty acid moieties, fatty amines and ethoxylated/propoxylated fatty amines.

Preferably, the cationic surfactant is a mono-long alkyl tri short alkyl quaternized ammonium salt or di-long alkyl di short alkyl quaternized ammonium salt wherein one or two alkyl substituents are selected from an aliphatic group of from about 8 to about 30 carbon atoms or an aromatic, alkoxy, polyoxyalkylene, alkylamido, hydroxyalkyl, aryl or alkylaryl group having up to about 30 carbon atoms; the other alkyl groups are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms; and the counter ion is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. The aliphatic groups can contain, in addition to carbon and hydrogen atoms, ether linkages, and other groups such as amino groups. The longer chain aliphatic groups, e.g., those of about 8 carbons, or higher, can be saturated or unsaturated.

Preferably, one alkyl group is selected from an alkyl group of from about 8 to about 30 carbon atoms, more preferably from about 14 to about 26 carbon atoms, still more preferably from about 14 to 22 carbon atoms; the other alkyl groups are independently selected from the group consisting of —CH₃, —C₂H₅, —C₂H₄OH, —CH₂C₆H₅, and mixtures thereof; and the counter ion is selected from the group consisting of Cl—, Br, CH₃OSO₃—, and mixtures thereof. It is believed that such mono-long alkyl quaternized ammonium salts can provide, in addition to their emulsification capability, improved slippery and slick feel on wet hair, compared to multi-long alkyl quaternized ammonium salts. It is also believed that mono-long alkyl quaternized ammonium salts can provide improved hydrophobicity and smooth feel on dry hair, compared to amine or amine salt cationic surfactants.

Nonlimiting examples of such mono-long alkyl quaternized ammonium salt cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; stearyl trimethyl ammonium chloride available, for example, with tradename CA-2450 from Nikko Chemicals; cetyl trimethyl ammonium chloride available, for example, with tradename CA-2350 from Nikko Chemicals; behenyltrimethylammonium methyl sulfate, available from FeiXiang; hydrogenated tallow alkyl trimethyl ammonium chloride; stearyl dimethyl benzyl ammonium chloride; and stearoyl amidopropyl dimethyl benzyl ammonium chloride.

Preferred cationic surfactants are saturated or unsaturated fatty acid based mono-ester and di-ester quats (quats=quaternary ammonium cation comprising compound) having 10 to 18 carbon atoms in the alkyl chain. Commercially available examples are Arquad PC SV-60 PG and Armocare VGH70 (Akzo Nobel).

Details on cationic surfactants are disclosed in US2013/259820.

The aqueous compositions of the present invention preferably comprise the cationic surfactant in amount of from about 0.05% to about 15%, preferably from about 0.05% to about 5%, still more preferably from about 0.1% to about 5%, specifically from 0.1 to 3% by weight, alternatively 0 wt % of the composition.

Preferred examples for nonionic emulsifiers are ethylene oxide (EO), propylene oxide (PO) and butylene oxide (BO) containing linear or branched C8 to C50, preferred C8 to 40, more preferred C8 to C24 fatty alcohol and fatty acid based emulsifiers as well as saccharide based emulsifiers, i.e. alkyl glycosides, alkoxylated fatty acid sorbitane esters and fatty acid glucamides. Another variety of preferred nonionic surfactants are the semi-polar amine oxides, phosphine oxides, and sulfoxides.

Preferred nonionic surfactants are saturated or unsaturated natural alcohol based ethoxylates having 10 to 18 carbon atoms in the alkyl chain and 5 to 80 EO units. Commercially available examples are the Genapol C, LA, V, O and T types (Clariant).

Preferred nonionic surfactants are linear or branched oxo alcohol based ethoxylates having 11 to 17 carbon atoms in the alkyl chain and 5 to 100 EO units. Commercially available examples are the Genapol UD, OA, OX, X, LCN types (Clariant).

Preferred nonionic surfactants are saturated or unsaturated alcohol based block ethoxylates-propoxylates having 10 to 18 carbon atoms in the alkyl chain and 2 to 20 EO units. Commercially available examples are the Genapol EP types (Clariant).

Preferred nonionic surfactants are ethoxylate-propoxylate block copolymers containing 5 to 70% wt % EO units. Commercially available examples are the Genapol PF and PH types (Clariant).

Preferred nonionic surfactants are saturated or unsaturated fatty acid based ethoxylates having 10 to 18 carbon atoms in the alkyl chain and 5 to 100 EO units. Commercially available examples are the Genagen O and S types (Clariant).

Preferred nonionic surfactants are saturated or unsaturated fatty acid based castor oil ethoxylates having 10 to 18 carbon atoms in the alkyl chains and 5 to 80 EO units. Commercially available examples are the Emulsogen HCO and EL types (Clariant).

Preferred nonionic surfactants are saturated or unsaturated fatty acid derivatized oligoglycerines. Preferred examples are fatty acid derivatized di-, tri, or tetraglycerines, i.e. mono- or diesters of diglycerine having having 10 to 18 carbon atoms in the alkyl chain and optionally 5 to 100 EO units. Commercially available examples are the Hostacerine types (Clariant).

Preferred nonionic surfactants are saturated or unsaturated fatty acid sorbitane ester based ethoxylates having 10 to 18 carbon atoms in the alkyl chain and 5 to 50 EO units attached to the sorbitane ring. A commercially available example is Emulsogen 4156 (Clariant).

Preferred nonionic surfactants are saturated or unsaturated alcohol based glycosides having 8 to 18 carbon atoms in the alkyl chain and 1 to 10 glycosyl units. Commercially available examples are Plantacare 818up and 1200up (BASF).

Preferred nonionic surfactants are saturated or unsaturated fatty acid based glucamides, preferred fatty acid N-methylglucamides, having 8 to 18 carbon atoms in the alkyl chain. A commercially available example is the MEGA-10 type (Avanti).

Preferred nonionic surfactants are saturated or unsaturated fatty acid based alkanolamides, preferred fatty acid based ethanolamides, having 8 to 18 carbon atoms in the alkyl chain. Commercially available examples are the Aminon C types (Kao).

Preferred nonionic surfactants are the fatty amine or fatty acid amide based amine oxides having 8 to 30 carbon atoms in the alkyl chain. Commercially available examples are the Tomamine AO types (Air products) and the Genamineox types (Clariant).

The aqueous compositions of the present invention preferably comprise the nonionic surfactant in amount of from about 0.05% to about 15%, preferably from about 0.05% to about 5%, still more preferably from about 0.1% to about 5%, specifically from 0.1 to 3% by weight, alternatively 0 wt % of the composition.

Preferred examples for betaine emulsifiers are carbobetaine, sulfobetaine, phosphatobetaine and phosphonatobetaine groups containing linear or branched C8 to C50, preferred C8 to 40, more preferred C8 to C30 alkyl, fatty alcohol and fatty acid based emulsifiers, i.e. cocoamidopropyl carbobetaines.

Preferably, suitable betaine surfactants for use in compositions according to the invention include those which are known for use in shampoo or other personal care cleansing. They include those surfactants broadly described as derivatives of aliphatic secondary and tertiary amines in which the aliphatic radical can be straight or branched chain and wherein one of the aliphatic substituents contains from about 8 to about 30 carbon atoms and one contains an anionic group such as carboxy, sulfonate, sulfate, phosphate, or phosphonate. Exemplary amphoteric surfactants for use in the formulations of the present invention include cocoamphoacetate, cocoamphodiacetate, lauroamphoacetate, lauroamphodiacetate, and mixtures thereof. They also include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 30 carbon atoms and one contains an anionic group such as carboxy, sulfonate, sulfate, phosphate or phosphonate.

Preferred carbobetaine surfactants are saturated or unsaturated fatty acid based sarcosides having 10 to 18 carbon atoms in the alkyl chain. A commercially available example is Medialan LD (Clariant).

Preferred carbobetaine surfactants are saturated or unsaturated fatty acid based am ido propyl betaines having 10 to 18 carbon atoms in the alkyl chain. A commercially available example is Genagen CAB (Clariant).

Preferred sulfobetaine surfactants are saturated or unsaturated fatty acid based taurides having 10 to 18 carbon atoms in the alkyl chain. A commercially available example is Hostapon CT (Clariant).

Details on betaine surfactants are disclosed in US2015/011449.

The compositions of the present invention preferably comprise the betaine surfactant in amount of from about 0.05% to about 15%, preferably from about 0.05% to about 5%, still more preferably from about 0.1% to about 5%, specifically from 0.1 to 3% by weight, alternatively 0 wt % of the composition.

Preferred examples for anionic emulsifiers are carboxylate, sulfate, sulfonate, phosphate and phosphonate groups containing linear or branched C8 to C50, preferred C8 to 40, more preferred C8 to C24 alkyl, fatty alcohol and fatty acid based emulsifiers, i.e. C8 to C24 fatty acid carboxylates, C8 to C24 fatty acid polyether carboxylates, C8 to C24 fatty acid polyether sulfates, C8 to C24 maleic acid addition products, C8 to C24 fatty alcohol sulfates, C8 to C24 sulfonates, C8 to C40 phosphates containing one or two fatty acid moieties.

Preferably, anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Exemplary anionic surfactants for use in the shampoo composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment of the present invention, the anionic surfactant is sodium lauryl sulfate or sodium laureth sulfate (sodium lauryl ether sulfate).

Preferred anionic surfactants are saturated or unsaturated fatty alcohol based polyether sulfates having 10 to 18 carbon atoms in the alkyl chain and 2 to 30 EO units. Commercially available examples are the Emulsogen EPM types (Clariant).

Preferred anionic surfactants are saturated or unsaturated fatty alcohol based polyether carboxylates having 10 to 18 carbon atoms in the alkyl chain and 2 to 30 EO units. Commercially available examples are the Empicol types (Huntsman).

Details on anionic surfactants are disclosed in US2015/011449.

Soaps include in particular salts of fatty acids such as alkaline or earth alkaline metal salts, such as sodium or potassium or calcium salts of C6 to C22 fatty acids, such as those obtained from saponification of triglycerides, e.g. alkaline or earth alkaline metal salts of lauric acid, myristic acid, palmitic acid, stearic acid, oleic acid, linoleic acid, linolenic acid.

The compositions of the present invention preferably comprise the anionic surfactant in amount of from about 0.05% to about 15%, preferably from about 0.05% to about 5%, still more preferably from about 0.1% to about 5%, specifically from 0.1 to 3% by weight, alternatively 0 wt % of the composition.

Further details on surfactants are disclosed in US 2009-0165812.

Preferred examples for silicone based emulsifiers are cationic, nonionic, betaine and anionic emulsifiers.

Preferred examples for cationic emulsifiers are quaternary ammonium groups containing emulsifiers of the ABA type with EO/PO moieties attached to the terminal quat (quaternary ammonium cation comprising) ends of a silicone chain (WO2009/042083) or quaternized emulsifiers having polyether moieties attached to the silicone chain in a comb like arrangement (US2008/213208).

In another preferred embodiment of the invention hydrophilic polyhydroxy moieties as well as oleophilic fatty alkyl or fatty alkyl ester moieties are attached to the silicone chain (US2012/289649). A commercially available example for this type of W/O emulsifier is Silform® EOF (available from Momentive Performance Materials).

The compositions of the present invention preferably comprise the silicone based cationic surfactant in amount of from about 0.05% to about 15%, preferably from about 0.05% to about 5%, still more preferably from about 0.1% to about 5%, specifically from 0.1 to 3% by weight of the composition.

Preferred examples for siloxane based nonionic emulsifiers are ethylene oxide (EO), propylene oxide (PO) and butylene oxide (BO) containing emulsifiers of the ABA type with EO/PO/BO moieties attached to the terminal ends of a silicone chain or emulsifiers having polyether moieties attached to the silicone chain in a comb like arrangement. A commercially available example is SF 1540 (available from Momentive Performance Materials). In another preferred embodiment of the invention, hydrophilic polyether moieties as well as oleophilic alkyl chains are attached to the silicone chain (U.S. Pat. No. 4,698,178). In another preferred embodiment of the invention, hydrophilic polyglycerol moieties as well as alkyl or fatty alcohol ether/fatty acid ester moieties are attached to the silicone chain (US2010/0266651, US2009/0062459). In another preferred embodiment of the invention amodimethicone glycerocarbamates are used (Dr. Frederic Pilz, COSSMA (2010) vol. 7-8 p18 and WO 2013017260 A1). In another preferred embodiment of the invention, cetyl diglyceryl tris(trismethylsiloxy)silylethyl dimethicones are used

(http://ec.europa.eu/consumers/cosmetics/cosing/index.cfm?fuseaction=search.details_v2&id=92003).

The latter four types of emulsifier are especially preferred for W/O emulsions.

The compositions of the present invention preferably comprise the silicone based nonionic surfactant in amount of from about 0.05% to about 15%, preferably from about 0.05% to about 5%, still more preferably from about 0.1% to about 5%, specifically from 0.1 to 3%, alternatively 0 wt % by weight of the composition.

The compositions of the present invention preferably comprise the silicone based betaine and anionic surfactant in amount of from about 0.05% to about 15%, preferably from about 0.05% to about 5%, still more preferably from about 0.1% to about 5%, specifically from 0.1 to 3%%, alternatively 0 wt % by weight of the composition.

It is within the scope of the invention to use more than one surfactant in order to optimize the formulation stability. The total amount on surfactants in the compositions of the present invention preferably ranges from about 0.05% to about 15%, preferably from about 0.05% to about 5%, still more preferably from about 0.1% to about 5%, specifically from 0.1 to 3% by weight, alternatively 0 wt % of the composition.

In a further embodiment of the invention the aqueous compositions optionally comprise additional additives, such as

a) organic diluents or solvents,
b) proteins, preferably keratin,
c) emollients or fatty substances,
d) preservatives,
e) skin protecting ingredients,
f) conditioning agents,
g) oxidizing agents,
h) reducing agents,
i) tannins,
j) metal salts,
k) further auxiliaries selected from pH adjusting agents, thickeners, lipids, amino acids, sugars, fragrances, sunscreen agents, vitamins, pearlescent agents, gelling agents, trace elements, sequestering agents, antioxidants, humectants, anti-hair loss agents, anti-dandruff agents, propellants, ceramides, polymers, in particular film-forming polymers, fillers, nacres, colorants, in particular pigments and dyes, and mixtures thereof,
with the proviso that oxidizing agents and reducing agents are not present simultaneously in a given composition.

Preferably, the aqueous compositions and also the cosmetic compositions of the invention comprise the following components:

| Ingredient | All figures are weight- % |
|---|---|
| at least one compound of the formulas | 0.05 to 30, preferably 1 to 10 |
| R²(—F)₂₋₁₈, | |
| R^{2′}(—F)₂₋₁₈, or | |
| R^{2″}(—F)₂₋₁₈ | |
| each as defined above, | |
| hydrocarbon or silicone based surfactant | 0 to 15, preferably 0.05 to 5 |
| in particular as described before | |
| water | q.s. to add to 100 |
| diluents/solvents | 0 to 95, preferably 0.1 to 95, preferred 10 to |
| | 95, more preferred 20 to 95, even more |
| | preferred 20 to 50 and 50 to 95 |
| protein, preferred keratin | 0 to 15, preferably 0 to 10, such as 0.01 to 5 |
| emollients/fatty substance | 0 to 15, preferably 0 to 10, more preferred 0 to |
| | 5, such as 0.01 to 5 |
| preservatives | 0 to 5, preferably 0 to 3, more preferred 0 to 2, |
| | such as 0.01 to 2.5 |
| skin protecting ingredients | 0 to 15, preferred 0 to 10, more preferred 0 to |
| | 5, even more preferred 0 to 1, specifically 0 to |
| | 0.1, e.g. 0.01 to 5 |
| conditioning agents | 0 to 15, preferred 0 to 10, more preferred 0 to |
| | 5, even more preferred 0 to 1, specifically 0 to |
| | 0.1, e.g. 0.01 to 5 |
| oxidizing agents | 0 to 15, preferred 0 to 10, more preferred 0 to |
| | 5, even more preferred 0 to 2, e.g. 0.01 to 5 |
| reducing agents | 0 to 15, preferred 0 to 10, more preferred 0 to |
| | 5, even more preferred 0 to 2, e.g. 0.01 to 5 |
| tannins | 0 to 15, preferred 0 to 10, more preferred 0 to |
| | 5, even more preferred 0 to 2, e.g. 0.01 to 5 |
| metal salts, such iron or zinc salts, | 0 to 15, preferred 0 to 10, more preferred 0 to |
| | 5, even more preferred 0 to 2, e.g. 0.01 to 5 |
| hair dyeing agent | 0 to 15, preferred 0 to 10, more preferred 0 to |
| | 5, even more preferred 0 to 2, e.g. 0.01 to 5 |
| further auxiliary agents | 0 to 15, preferred 0 to 10, more preferred 0 to |
| | 5, even more preferred 0 to 2, e.g. 0.01 to 5 |

wherein the wt-percentages relate to the complete weight of the aqueous compositions, and the individual wt-ranges may relate to a single component of the said class of components, but preferably relates to the total weight of each components of the said class of components.

### Diluents/Solvents

The term “diluents/solvents” refers to substances that may be used to dilute/solvatize the at least one polyorganosiloxane A) and/or the at least one organic compound B) according to the invention and the other optional other ingredients as mentioned before in addition to water. Suitable organic solvents are i.e. 2-methyl-1,3-propanediol, mono and dialcohols or the ethers and esters thereof, in particular mono-C1-C3-alkyl ether, ethanol, n-propanol, isopropyl alcohol, tert. butanol, 1-methoxypropanol, 1-ethoxypropanol and ethoxydiglycol, diols and their ethers and esters, 1,3 propanediol, 1,3- and 1,4-butanediol, pentylene glycol, hexylene glycol, diethyleneglycol and the monomethyl and monoethyl ether thereof, dipropylene glycol and the monomethyl and monoethyl ether thereof, glycerol, diglycerol, hexanetriol, sorbitol, ethyl carbitol, benzyl alcohol, benzyloxy ethanol, propylene carbonate, N-alkyl pyrrolidone. In a preferred embodiment water/ethanol, water/isopropyl alcohol, water/dipropylene glycol and water propylene glycol mono methyl ether mixtures are used. Generally, the addition of certain amounts of short chained alcohols improves the homogeneity of the formulations and the penetration of the formulations into the hair. Depending on the polymer structure type and the application purpose certain quantities on acids, bases and/or short chained alcohols are required in order to get transparent formulations. Suitable acids include inorganic or organic acids, like for example carboxyl acids, like acetic acid, hydrochloric acid, sulfuric acid, phosphoric acid. Suitable bases include aqueous ammonia, alkaline hydroxides, alkaline carbonates, etc.

### Protein/Keratin

The optional protein, preferred keratin protein fractions used comprise hydrolyzed keratin produced by alkaline and/or enzymatic hydrolysis using methods known in the art. The keratin hydrolysate is about 1,000-3,000 molecular weight. The keratin may be derived from human or other mammalian sources such as goat hair (US 2007-0048235), hoof or horn meals, (U.S. Pat. No. 6,555,505). Alternatively, “keratin protein fraction” is a purified form of keratin that contains predominantly, although not entirely, one distinct protein group as described in U.S. Pat. No. 7,148,327. Details on the keratin and keratin fractions are disclosed in US 2009-0165812.

### Emollients, Fatty Substances

A further optional ingredient of the hair treatment formulations is one or more emollients. An “emollient” is a material that protects against wetness or irritation, softens, soothes, supples, coats, lubricates, moisturizes, protects and/or cleanses the skin. Emollients used comprise one or more of: a silicone compound, i.e. dimethicones, cyclomethicones, preferred D₅ and D₆ cyclosiloxanes, dimethicone copolyols or mixtures of cyclomethicones and dimethicone/vinyldimethicone cross polymer), polyols such as sorbitol, glycerin, propylene glycol, ethylene glycol, polyethylene glycol, caprylyl glycol, polypropylene glycol, 1,3-butane diol, hexylene glycol, isoprene glycol, xylitol, ethylhexyl palmitate, a triglyceride such as caprylic/capric triglyceride and fatty acid ester such as cetearyl isononanoate or cetyl palmitate. Details on emollients are disclosed in US 2009/0165812.

As fatty substances that are liquid at ambient temperature, often referred to as oils, that can be used in the invention, mention may be made of: hydrocarbon-based oils of animal origin, such as perhydrosqualene, hydrocarbon-based plant oils, such as liquid triglycerides of fatty acids containing 4 to 10 carbon atoms, for instance heptanoic or octanoic acid triglycerides, or else sunflower oil, maize oil, soya oil, grapeseed oil, sesame oil, apricot oil, macadamia oil, castor oil, avocado oil, caprylic/capric acid triglycerides, jojoba oil, shea butter; linear or branched hydrocarbons of mineral or synthetic origin, such as liquid paraffins and derivatives thereof, petroleum jelly, polydecenes, hydrogenated polyisobutene such as Parleam®; synthetic esters and ethers, in particular of fatty acids, for instance purcellin oil, isopropyl myristate, 2-ethylhexyl palmitate, 2-octyldodecyl stearate, 2-octyldodecyl erucate, isostearyl isostearate, hydroxylated esters, for instance isostearyl lactate, octyl hydroxystearate, octyldodecyl hydroxystearate, diisostearyl malate, triisocetyl citrate, fatty alcohol heptanoate, octanoate and decanoate; polyol ester, for instance propylene glycol dioctanoate, neopentyl glycol diheptanoate, diethylene glycol diisononanoate, pentaerythritol esters, fatty alcohols having 12 to 26 carbon atoms, for instance octyldodecanol, 2-butyloctanol, 2-hexyldecanol, 2-undecyl pentadecanol, oleyl alcohol, partially hydrocarbon-based and/or silicone-based fluoro oils, silicone oils, for instance volatile or non-volatile, linear or cyclic polydimethylsiloxanes (PDMS) which are liquid or pasty at ambient temperature (25° C.), such as cyclomethicones, dimethicones, optionally comprising a phenyl group, for instance phenyl trimethicones, phenyl trimethylsiloxydiphenylsiloxanes, diphenylmethyl- dimethyltrisiloxanes, diphenyl dimethicones, phenyl dimethicones , polymethylphenylsiloxanes; mixtures thereof. Details on suitable fatty substances are disclosed in WO 2012-038334.

### Preservatives

Optionally, one or more preservatives may be included in the hair treatment formulations. Examples of such preservatives comprise one or more glycerin containing compound (e.g., glycerin or ethylhexylglycerin or phenoxyethanol), lactic acid, benzyl alcohol, EDTA, potassium sorbate and/or grapefruit seed extract. In a preferred embodiment, the hair straightening formulations are paraben free. Details on preservatives are disclosed in US 2009/0165812.

### Skin Protecting Agents

Optionally, the hair treatment formulations comprise one or more skin protecting agents. Skin protecting agents comprise one or more agents that prevent the undesired transmission of microbes or organic/inorganic chemicals. Details on skin protecting agents are disclosed for examples in US 2009/0165812.

### Conditioning Agents

Optionally, one or more conditioning agent may be included in the hair treatment formulations. In one preferred embodiment silicone based conditioning agents are incorporated. Preferred materials are PDMS grades ranging from 10 to 1.000.000 mPa·s, C2 to C18-alkyl derivatized silicones, dimethiconols, polyether modified silicones, amino groups or quaternized ammonium groups containing silicones. They may be also selected from polyorganosiloxanes having functional groups FA as defined above. These silicones can be incorporated as neat materials, organic solutions, emulsions or microemulsions.

Preferred examples for quaternary ammonium groups (quats) containing conditioning agents are α,ω-quat group terminated silicones (U.S. Pat. No. 4,891,166), quat group terminated T shaped silicones (US2008027202), α,ω-silicone block terminated quats (WO02/10256) and silicones containing quat groups in a comb like arrangement, optionally containing additional moieties, i.e. polyethers or aromatic structures (US2008213208, U.S. Pat. Nos. 5,098,979, 5,153,294, 5,166,297, US2006188456). Other preferred examples are quat group/silicone block based copolymers (EP282720, U.S. Pat. Nos. 6,240,929, 6,730,766, DE102004002208). In another preferred embodiment quat group/silicone block/hydrophilic block based copolymers are used (WO 02/10257 and WO 02/10259, U.S. Pat. Nos. 7,563,856, 7,563,857, US20110039948, US2007106045, US2005255073, WO2004069137). Other preferred examples are quat group/silicone block based copolymers and quat group/silicone block/hydrophilic block based copolymers bearing terminal monofunctional silicone moieties (WO2013148629, WO2013148635, WO2013148935). In another preferred embodiment of the invention quat group terminated silicones bearing pending amino groups are used (DE10253152). Other preferred examples are silicone betaines (DE10036522, DE10036532). Commercially available examples for quaternary ammonium groups containing siloxanes are Silsoft Silk and Silsoft Q (available from Momentive Performance Materials).

The above described silicone based conditioning agents in particular impart a smooth and silky feel to hair.

Alternatively, hydrocarbon based conditioning agents can be included. Details on these cationic types of material, containing amino and/or quaternary ammonium groups are disclosed for example in US 2009/0000638 and WO 2012/027369.

### Oxidizing Agents

Optionally, one or more oxidizing agent may be included in the hair treatment formulations.

Preferred oxidizing agents include organic oxidizers, i.e. benzoquinone, other quinone derivatives including hydroquinone and aminoquinones and suitable organic peroxides. Details on organic oxidizers are disclosed in US 2012/0031420 and WO 2012/027369.

Hydrogen peroxide is the preferred inorganic oxidizing agent. Persulfates, in the form of their sodium potassium and ammonium salts, may also be used alone or in combination with the hydrogen peroxide just before use. Other possible oxidizing agents include sodium percarbonate, sodium perborate, magnesium perborate, magnesium dioxide and barium dioxide. Details on these oxidizing agents are disclosed in U.S. Pat. No. 6,544,499.

### Reducing Agents

Optionally, one or more reducing agent may be included in the hair treatment formulations with the proviso that oxidizing agents and reducing agents are not present simultaneously in a given formulation. Preferred reducing agents are thioglycolic acid and thiolactic acid as well as the salts thereof, in particular the ammonium and ethanolamine salts. Further useful thio compounds are in particular cysteine or the hydrochloride thereof, homocysteine, cysteamine, N-acetyl cysteine, thioglycerol, ethanediol monothioglycollate, 1,2-propyleneglycol monothioglycollate (see also WO 93/1791), 1-3-propanediol monothioglycollate or the isomer mixture resulting therefrom, 1,3-butanediol and 1,4-butanediol monothioglycollate and the isomer mixtures therefrom, polyethylene glycol, such as di-, tri- and tetraethyleneglycol monothioglycollates, glycerol monothiolactate and further thio acids and the esters thereof, as well as mixtures thereof. Details on these organic reducing agents are disclosed in US 2009/0000638.

The usage of inorganic reducing sulfur compounds is basically also possible. Representative examples for use in the reducing compositions include cosmetically acceptable salts (e.g., alkali metal (e.g., sodium and potassium) and ammonium salts), esters (e.g., lower alkyl amines (e.g., triethanolamine (TEA), monoethanolamine (MEA) and aminomethyl propanol (AMP), of sulfite, disulfite, bisulfite, metabisulfite, hydrosulfite, hyposulfite and pyrosulfite). Specific examples of suitable reducing agents thus include sodium metabisulfite, potassium metabisulfite, sodium sulfite, potassium sulfite, sodium thiosulfate, potassium thiosulfate, ammonium bisulfite, ammonium sulfite, ammonium metabisulfite, MEA sulfite, MEA metabisulfite, potassium bisulfite, sodium bisulfite, ammonium bisulfite, sodium hydrosulfite, potassium hydrosulfite, ammonium hydrosulfite, anhydrous sodium sulfite, diammonium sulfite, dipotassium disulfite, dipotassium pyrosulfite, AMP sulfite, AMP metabisulfite, TEA sulfite, TEA metabisulfite, sodium acid sulfite, sodium hyposulfite, sodium pyrosulfite, and sodium thiosulfate pentahydrate. Details on these inorganic reducing agents are disclosed in WO 2012/027369.

Alternatively, high temperature and alkali-treated keratin, wherein the keratin is heated to around 100° C. or above, dithionites and certain hydrides can be used. Details on these reducing agents are disclosed in U.S. Pat. No. 6,544,499.

### K) Tannins

Optionally one or more tannins, specifically gallotannins, ellagitannins, complex tannins, condensed tannins, i.e. tannic acid and its other forms quercitannic acid and gallotannic acid may be used. Tannins represent a class of polyphenol derivatives and are known for their structural diversity. A classification is given based on K. Khanbabaee, T. van Ree, Nat. Prod. Rep., 2001, 18, 641-649 which is herewith included by reference and used to define the term tannins in the context of the present invention. The most preferred tannin is gallotannic acid (=tannic acid). Preferred tannins include:

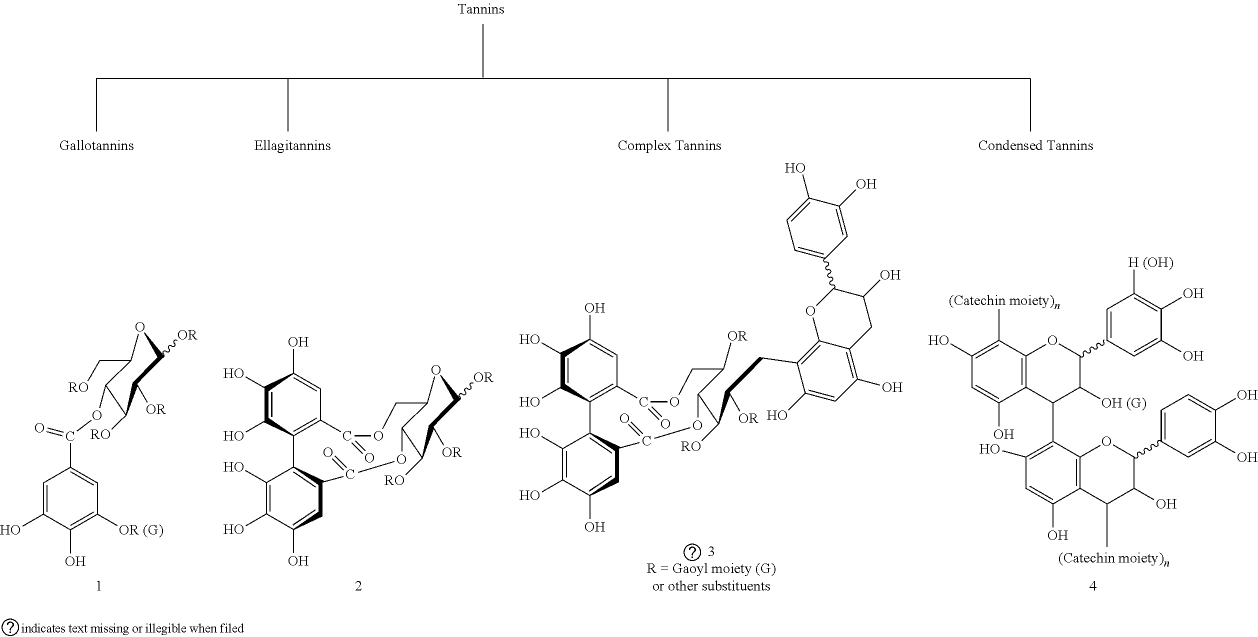

Examples for gallotannins are

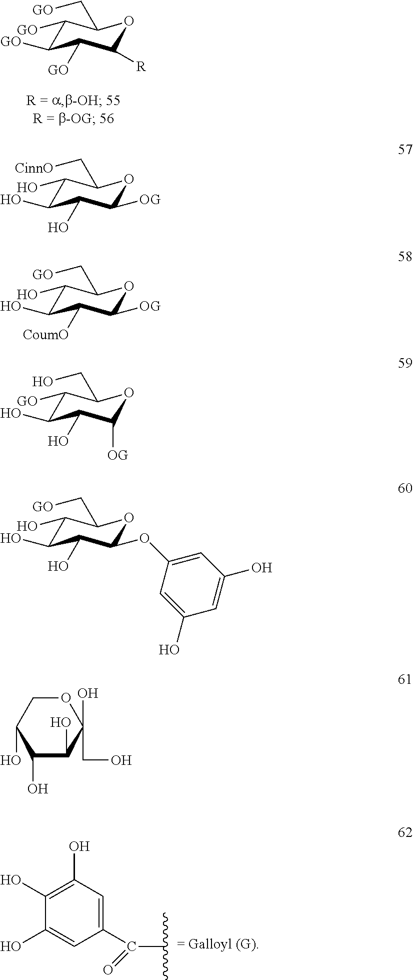

Examples for ellagitannins are

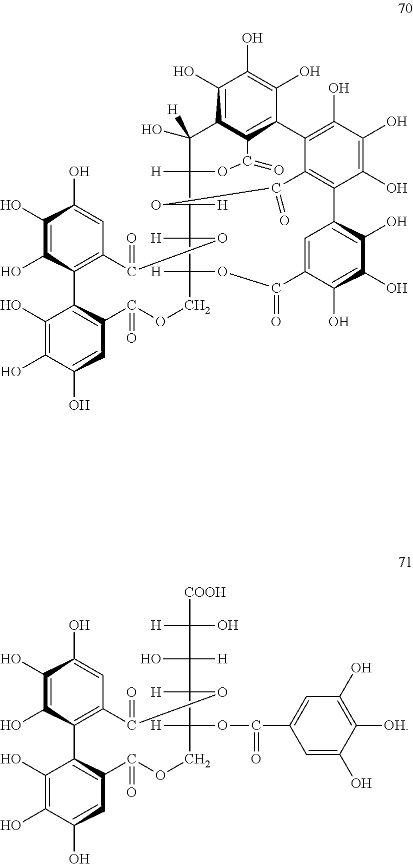

An example for a complex tannin is Acutissimin A

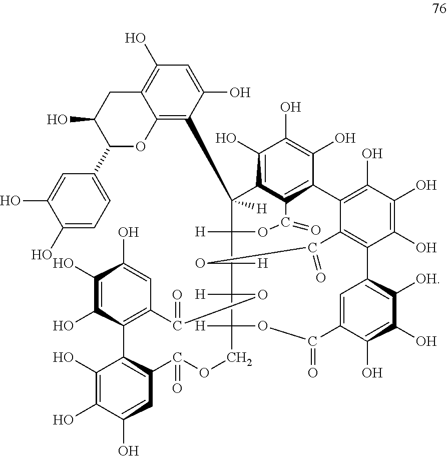

Examples for condensed tannins are procyanidin B2 (77), proanthocyanidin A1 (78), proanthocyanidin A2 (79) and proanthocyanidin C1 (80):

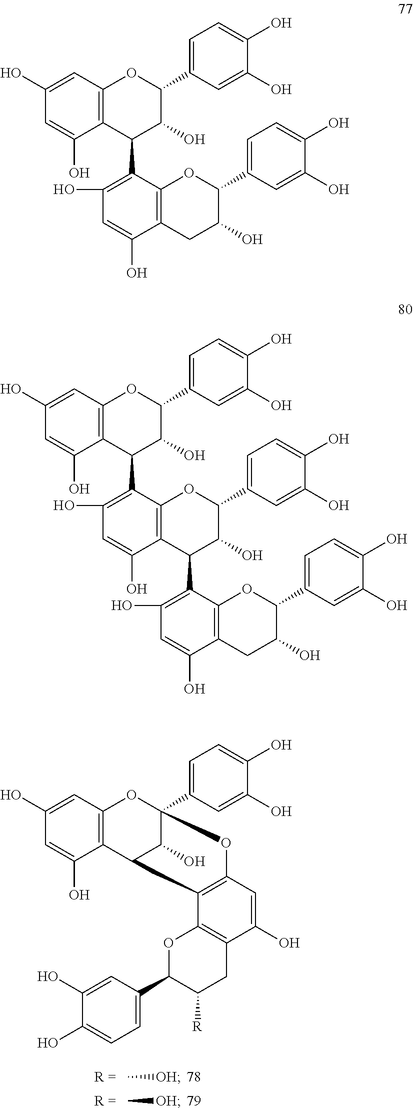

The most preferred tannin is tannic acid:

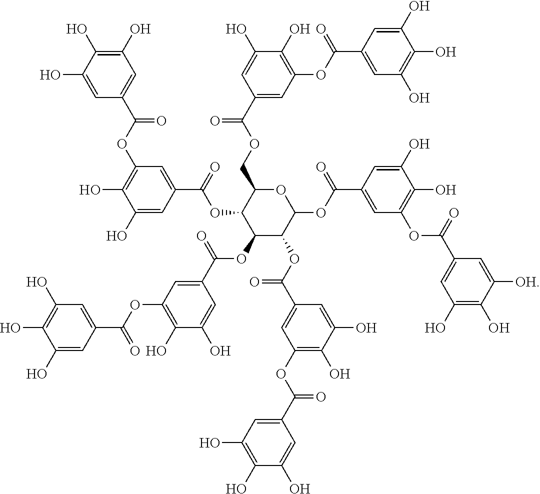 [SMILES: O=C(OC[C@H]1OC(OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1]

### Metal Salts

Include in particular those of general formula:

MeₓA_{y}

wherein Me in this formula is a metal cation and the number of cations Me is x and the number of anions A is y and the numbers x and y are such that the salt is neutral. x may be e.g. 1 or 2, y may be e.g. 1 to 3 in particular. A is preferably (i) the anion of an oxidized carbohydrate of the formula —O—C(O)—R, or an anion derived from an inorganic or organic acid. Me is preferably an iron or zinc cation.

Particular preferred salts are Fe or Zn salts, which are preferably water-soluble, such as Fe²⁺ lactobionate, Fe²⁺ maltobionate, Fe²⁺ isomaltobionate, Fe³⁺ lactobionate, Fe³⁺ maltobionate, Fe³⁺ isomaltobionate, Fe²⁺ gluconate, Fe³⁺ gluconate, Fe²⁺ glucoheptonate, Fe³⁺ glucoheptonate, Fe²⁺ glycerophosphate, Fe³⁺ glycerophosphate, Zn²⁺ lactobionate, Zn²⁺ maltobionate, Zn²⁺ isomaltobionate, Zn²⁺ gluconate, and Zn²⁺ glycerophosphate, Fe²⁺ tartrate, Fe²⁺ glucarate, Fe³⁺ tartrate, Fe³⁺ glucarate, Zn²⁺ tartrate, Zn²⁺ glucarate.

The weight ratio of the Fe or Zn salt to the compound according to the invention containing the di- or trihydroxy-substituted aromatic groups is at least 0.01, preferred 0.01 to 3, more preferred 0.,02 to 3, even more preferred 0.05 to 3, specifically 0.1 to 3, even more specific 0.1 to 1. If the optional tannins are used in addition then the weight ratio of the water soluble Fe or Zn salt to the compound containing di- or trihydroxy-substituted aromatic groups according to the invention+tannins is at least 0.01, preferred 0.01 to 3, more preferred 0.02 to 3, even more preferred 0.05 to 3, specifically 0.1 to 3, even more specific 0.1 to 1. If the optional tannins are used then the weight ratio of the tannins to the organic compound containing di- or trihydroxy-substituted aromatic groups according to the invention is 0.01 to 5, preferred 0.01 to 3, more preferred 0.02 to 3, even more preferred 0.05 to 3, specifically 0.1 to 3, even more specific 0.1 to 1.

The specific amount on water soluble Fe or Zn salt depends on the molecular weight and the structure of the water soluble Fe or Zn salt as well as on molar ratio of the di- or trihydroxy-substituted aromatic groups in the inventive compound and the optionally used tannins. Typically, the higher the molecular weight of the water soluble Fe or Zn salt and/or the higher the molar ratio of the di- or trihydroxy-substituted aromatic groups in the inventive compounds and the optional tannins the higher the specific amount on water soluble Fe or Zn salt used.

### Further Auxiliaries

The hair treatment formulations may also comprise one or more additional auxiliaries, i.e. pH adjusting agents, such acids, bases and buffers to adjust the pH value, thickeners (such as polysaccharide thickeners, starch, modified starches, xanthan, gellan, carragenan, pullulan, cellulose, cellulose derivatives, polyacrylic acids, polyacrylates copolymers, polyacrylamides, pectins, clays, fumed silica), lipids, amino acids, sugars, fragrances, sunscreen agents, vitamins, pearlescent agents, gelling agents, trace elements, sequestering agents, antioxidants, humectants, anti-hair loss agents, anti-dandruff agents, propellants, ceramides, polymers, in particular film-forming polymers; fillers, nacres, colorants and in particular pigments and dyes, including hair dyeing agents as described below, all kinds of bioactive phytochemicals, and also mixtures thereof.

### Hair Dyeing Agents

Hair dyeing agents include commonly used oxidative or non-oxidative, temporary, semipermanent, demipermanent and permanent hair dyes. Temporary non-oxidative dyes include e.g. Acid Yellow, Acid Orange 7, Acid Yellow 1, Acid Red 33, Acid Red 92, Acid Violet 43, Acid Blue 9, Acid Black 1, which are commonly used in mixtures. Semi-Permanent Non-Oxidative Hair Dyeing Agents contain basic or cationic dyes with low molar mass, and include in particular HO Yellow No. 2, HO Red No. 3, 4-hydroxypropylamino-3-nitrophenol, N,N-bis-(2-hydroxyethyl)-2-nitrophenylenediamine, HO Blue No. 2, Basic Red 51, Basic Red 76, Basic Brown 16, Basic Brown 17, Basic Blue 99, Basic Yellow 57. Other semipermanent dyes, include metallic and vegetables derivatives (such as Henna). The metallic dyes are derived from silver salts, lead, and bismuth. Permanent Oxidative Hair Dyeing Agents include commonly used complex systems of precursors in the presence of an oxidizing agent.

Depending on the polymer structure type and the application purpose certain quantities on acids, bases and/or short chained alcohols are required in order to get transparent formulations. Suitable acids include inorganic or organic acids, like for example carboxylic acids, like acetic acid, hydrochloric acid, sulfuric acid, and phosphoric acid. Suitable bases include aqueous ammonia, alkaline hydroxides, alkaline carbonates, etc.

By adding for example such acids or bases suitable pH ranges of the the aqueous compositions can be adjusted such as below 9, preferably below 8.5, preferably below 7.5, more preferably below 7.0.

A further preferred embodiment of the present invention relates to a cosmetic composition or personal care formulation comprising at least one compound according to the invention, or at least one aqueous composition according to the invention, each as defined herein.

Examples of personal care compositions or cosmetic compositions in which the compounds or the invention can be utilized include, but are not limited to, e.g. deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail-and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, sunscreens, cosmetics, hair care products, skin care products, toothpastes, drug delivery systems for topical application of medicinal compositions that are to be applied to the skin, combinations of two or more thereof, etc. Such cosmetic or personal care compositions of the present invention may include other ingredients and components as desired for a particular purpose or intended use. For example, personal care compositions may include ingredients chosen from emollient, moisturizer, humectant, pigment, coated mica, colorant, fragrance, biocide, preservative, antioxidant, anti-microbial agent, anti-fungal agent, antiperspirant agent, exfoliant, hormone, enzyme, medicinal compound, vitamin, salt, electrolyte, alcohol, polyol, absorbing agent for ultraviolet radiation, botanical extract, surfactant, silicone oil, organic oil, wax, film former, thickening agent, particulate filler, clay, surfactants, emulsifiers, solvents, emollients, moisturizers, humectants, pigments, colorants, fragrances, biocides, preservatives, chelating agents, antioxidants, anti-microbial agents, anti-fungal agents, antiperspirant agents, exfoliants, hormones, enzymes, medicinal compounds, vitamins, alpha-hydroxy acids, beta-hydroxy acids, retinols, niacinamide, skin lightening agents, salts, electrolytes, alcohols, polyols, absorbing agents for ultraviolet radiation, botanical extracts, organic oils, waxes, film formers, thickening agents, particulate fillers, silicones, clays, plasticizers, humectants, occlusive, sensory enhancers, esters, resins, film formers, film forming emulsifiers, high refractive index materials, combinations of two or more thereof, etc.

Cosmetic products that can be applied to the face such as skin-care creams, lipsticks, eye and facial makeup, towelettes, and colored contact lenses. Cosmetic products that can be applied to the body such as deodorants, lotions, powders, perfumes, baby products, bath oils, bubble baths, bath salts, and body butters; to the hands/nails: fingernail and toe nail polish, and hand sanitizer; to the hair: permanent chemicals, hair colors, hair sprays, and gels; makeup compositions comprising color pigments, cosmetic products that can applied in particular to the face and eye area such as primers, foundations or eyeshadows, lipsticks, lip gloss, lip liner, lip plumper, lip balm, lip stain, lip conditioner, lip primer, lip boosters, and lip butters which may contain sunscreens, concealers, face powders, mascara, eye shadow, eye liner, eyebrow pencils, creams, waxes, gels, and powders which are used to color, fill in, and define the brows; lotions, cleansing formulations; toners; facial masks; peel masks, sheet masks, exfoliant products, moisturizers such as creams or lotions which may contain essential oils, herbal extracts, or other chemicals; night creams, day creams, sunscreen compositions, nail polishes etc.

Personal care articles, which may comprise the compounds of the invention, include e.g. cosmetic and medical articles, such e.g. bar soap, liquid soap (e.g., hand soap), hand sanitizer (including rinse off and leave-on alcohol based and aqueous-based hand disinfectants), preoperative skin disinfectant, cleansing wipes, disinfecting wipes, body wash, acne treatment products, skin cream, shampoo, conditioner, cosmetics (including but not limited to liquid or powder foundation, liquid or solid eyeliner, mascara, cream eye shadow, tinted powder, “pancake” type powder to be used dry or moistened, etc.) deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream, Wound care items, such as, but not limited to, wound healing ointments, creams, and lotions, wound coverings, burn wound cream, bandages, tape, and steri-strips, and medical articles such as medical gowns, caps, face masks, and shoe-covers, surgical drops, etc. Additional products include but are not limited to oral products such as mouth rinse, toothpaste, and dental floss coatings, veterinary and pet care products, preservative compositions, and surface disinfectants including solutions, sprays or wipes, etc.

The aqueous or cosmetic compositions according to the invention can be (used in or) formulated, in particular, into a form typical for hair treatment compositions. Preferred are topical hair care or treatment compositions, e.g. hair tonics, conditioners, hair-care preparations, e.g. pre-treatment preparations, styling creams, styling gels, pomades, hair rinses, treatment packs, intensive hair treatments e. g. leave-on and rinse-off deep conditioners, hair-structuring preparations, e.g. hair-waving preparations for permanent waves (hot wave, mild wave, cold wave), hair-straightening preparations, liquid hair-setting preparations, hair foams, hair serums, hair sprays, bleaching preparations, e g. hydrogen peroxide solutions, lightening shampoos, bleaching creams, bleaching powders, bleaching pastes or oils, temporary, semi-permanent or permanent hair colorants, preparations containing self-oxidizing dyes, or natural hair colorants, such as henna or chamomile. Based on the application the hair care preparations may be in particular in the form of a (aerosol) spray, (aerosol) foam, gel, gel spray, cream, lotion, liquid, serum or a wax, mousse, shampoo, such as pearl shampoo, anti-frizz shampoo etc. The aqueous compositions according to the invention can be used as leave-on or rinse-off hair treatment compositions.

A particular preferred cosmetic composition according to the invention is for use in hair treatment, preferably for use in hair coloring treatment. The above described aqueous cosmetic compositions according to the invention can provide particularly benefits with respect to an improved durability of artificial colors on hair. In addition the aqueous hair treatment formulations according to the invention provide a hair strengthening and shaping effect as well as a conditioning effect, in particular, before, during and after a hair dyeing treatment, such as hair bleaching treatment. The hair treatment compositions according to the invention can provide benefits with respect to the strengthening of hair, the hair coloration, the color retention and the shaping of hair, i.e. the curling and straightening of hair.

Preferred cosmetic compositions for the treatment of hair according to the invention are selected from the group consisting of a hair shampoo composition, hair care composition, hair conditioning composition, hair strengthening composition, hair coloration or dyeing composition, hair combability improving composition, anti-frizz composition, hair rinse-off and leave-on compositions.

In a further embodiment the invention relates to a process for the treatment of hair which comprises the steps of providing a cosmetic composition according to the invention, and applying said cosmetic composition to said hair. Such process for the treatment of hair may further comprises the step of dyeing the hair.

A particular preferred process for the treatment of hair involves steps of:

1) contacting the hair with the aqueous cosmetic composition of the invention,
2) optionally contacting the hair with an aqueous composition containing a metal salt of Fe and/or Zn, comprising a water phase, having a pH ranging from >2 to 10, and containing water soluble Zn²⁺, Fe²⁺ and/or Fe³⁺ salts with counter ions preferably selected from
chloride, sulphate, alkylsulphonate, arylsulphonate, alkylarylsulphonate, hydroxy functionalized carboxylic acids, specifically selected from
C2 to C12, preferred C2 to C9 saturated or unsaturated hydroxyalkyl carboxylic acids, i.e. glycolic acid, lactic acid, β-hydroxy butyric acid, γ-hydroxy butyric acid, 2-hydroxy-succinic acid, citric acid, mandelic acid,
C2 to C12, preferred C2 to C9, also preferred C7 to C12, more preferred C7 to C9 saturated or unsaturated polyhydroxyalkyl carboxylic acids, i.e. glyceric acid, 2,2-dimethylolpropane carboxylic acid, arabinonic acid, gluconic acid, glucoronic acid, glucoheptonic acid, glucopyranosyl arabinonic acid, lactobionic acid, maltobionic acid, tartaric acid, glucaric acid,
C7 to C12, preferred C7 to C9 hydroxyaromatic carboxylic acids, i.e. 2-hydroxy benzoic acid, 3-hydroxy benzoic acid, 4-hydroxy benzoic acid, 2-hydroxy cinnamic acid, 3-hydroxy cinnamic acid, 4-hydroxy cinnamic acid,
C7 to C12, preferred C7 to C9 polyhydroxyaromatic carboxylic acids, i.e 2,3-dihydroxy benzoic acid, 2,4-dihydroxy benzoic acid, 2,5-dihydroxy benzoic acid, 2,6-dihydroxy benzoic acid, 3,4-dihydroxy benzoic acid, 3,5-dihydroxy benzoic acid, dihydroxy cinnamic acids or their partial esters, i.e. 3,4-dihydroxy cinnamic acid, trihydroxy benzoic acids or their partial esters, i.e. 2,3,4-trihydroxy benzoic acid, 2,3,5-trihydroxy benzoic acid, 2,3,6-trihydroxy benzoic acid, 2,4,5-trihydroxy benzoic acid, 2,4,6-trihydroxy benzoic acid, 3,4,5-trihydroxy benzoic acid, trihydroxy cinnamic acids or their partial esters, i.e. 3,4,5-dihydroxy cinnamic acid, and mixtures thereof, and
   3) Drying the hair at a temperature of preferably >150° C., for example during a hot ironing step, which permanently bonds to the hair or incorporates into the hair the inventive hair treatment compositions or parts of the hair treatment composition used in steps 1) and 2).
   Another particular preferred process for the treatment of hair involves steps of:
   1) contacting the hair with the aqueous cosmetic composition of the invention, comprising the compound of the invention, optionally one more surfactants as explained above, and a metal salt of Fe and/or Zn, preferably water-soluble Zn²⁺, Fe²⁺ and/or Fe³⁺ salts with counter ions preferably selected from
chloride, sulphate, alkylsulphonate, arylsulphonate, alkylarylsulphonate, hydroxy functionalized carboxylic acids, specifically selected from
C2 to C12, preferred C2 to C9 saturated or unsaturated hydroxyalkyl carboxylic acids, i.e. glycolic acid, lactic acid, β-hydroxy butyric acid, γ-hydroxy butyric acid, 2-hydroxy-succinic acid, citric acid, mandelic acid,
C2 to C12, preferred C2 to C9, also preferred C7 to C12, more preferred C7 to C9 saturated or unsaturated polyhydroxyalkyl carboxylic acids, i.e. glyceric acid, 2,2-dimethylolpropane carboxylic acid, arabinonic acid, gluconic acid, glucoronic acid, glucoheptonic acid, glucopyranosyl arabinonic acid, lactobionic acid, maltobionic acid, tartaric acid, glutaric acid
C7 to C12, preferred C7 to C9 hydroxyaromatic carboxylic acids, i.e. 2-hydroxy benzoic acid, 3-hydroxy benzoic acid, 4-hydroxy benzoic acid, 2-hydroxy cinnamic acid, 3-hydroxy cinnamic acid, 4-hydroxy cinnamic acid,
C7 to C12, preferred C7 to C9 polyhydroxyaromatic carboxylic acids, i.e 2,3-dihydroxy benzoic acid, 2,4-dihydroxy benzoic acid, 2,5-dihydroxy benzoic acid, 2,6-dihydroxy benzoic acid, 3,4-dihydroxy benzoic acid, 3,5-dihydroxy benzoic acid, dihydroxy cinnamic acids or their partial esters, i.e. 3,4-dihydroxy cinnamic acid, trihydroxy benzoic acids or their partial esters, i.e. 2,3,4-trihydroxy benzoic acid, 2,3,5-trihydroxy benzoic acid, 2,3,6-trihydroxy benzoic acid, 2,4,5-trihydroxy benzoic acid, 2,4,6-trihydroxy benzoic acid, 3,4,5-trihydroxy benzoic acid, trihydroxy cinnamic acids or their partial esters, i.e. 3,4,5-dihydroxy cinnamic acid, and mixtures thereof, and
   2) Drying the hair at >150° C., e.g. during a hot ironing step, which permanently bonds to the hair or incorporate into the hair the inventive hair treatment composition or parts of the hair treatment composition used in step 1).

In the above processes the weight ratio of the optional water soluble Fe and/or Zn salt to the compound containing di- or trihydroxy-substituted aromatic groups according to the invention is at least 0.01, preferred 0.01 to 3, more preferred 0.02 to 3, even more preferred 0.05 to 3, specifically 0.1 to 3, even more specific 0.1 to 1. If the optional tannins are used then the weight ratio of the water soluble Fe and/or Zn salt to the organic compound containing di- or trihydroxy-substituted aromatic groups+tannins is at least 0.01, preferred 0.01 to 3, more preferred 0.02 to 3, even more preferred 0.05 to 3, specifically 0.1 to 3, even more specific 0.1 to 1. The specific amount on water soluble Fe and/or Zn salt depends on the molecular weight and the structure of the water soluble Fe or Zn salt as well as on molar ratio of the di- or trihydroxy-substituted aromatic groups in the inventive compound and the optionally used tannins. Typically, the higher the molecular weight of the water soluble Fe or Zn salt and/or the higher the molar ratio of the di- or trihydroxy-substituted aromatic groups in the inventive organic compounds and the optional tannins the higher the specific amount on water soluble Fe or Zn salt used.

Preferably, process step 1), contacting the hair with the hair treatment compositions based on the compounds containing di-, trihydroxy-substituted aromatic groups and optionally surfactants, to form treated hair is carried out at a temperature and length of time sufficient to penetrate the fiber. Typically, process step 1) is carried out at 10 to 50° C., preferred at 20 to 50° C., even more preferred room temperature, for 5 to 120 min, preferred 5 to 60 min, even more preferred 10 to 40 min.

Preferably, process step 2), contacting the hair with the inventive hair treatment compositions based on water soluble Zn²⁺, Fe²⁺ and/or Fe³⁺ salts to form further treated hair is carried out at a temperature and length of time sufficient to penetrate the fiber. Typically, process step 2) is carried out at 10 to 50° C., preferred at 20 to 50° C., even more preferred room temperature, for 5 to 120 min, preferred 5 to 60 min, even more preferred 10 to 40 min.

Preferably, process step 3) drying the treated hair by applying heat with a temperature of >150° C., preferably is a hot ironing step which permanently bonds to the hair or incorporates into the hair the inventive hair treatment compositions or parts of the hair treatment compositions used in steps 1) and 2). The elevated temperature can result in chemical reactions of components of the compositions used in steps 1) and 2) with reactive moieties of the keratin fiber or the formation of complexes of components of the compositions used in steps 1) and 2). Preferred temperatures range from 150° C. to about 235° C., more preferred from 180° C. to about 225° C., even more preferred from 190° C. to about 215° C. Typically, 2 to 10 rounds of hot ironing are applied in case of drying the hair.

In the context of the present invention water soluble Zn and/or Fe salts used in step 2) preferably have a water solubility of at least 0.5 g/l at 25° C.

The above described hair treatment process according to the invention can provide benefits with respect to the strengthening of hair, the hair shaping, i.e. the curling and straightening of hair, the hair coloration, the hair bleaching, the hair color retention, the hair conditioning.

The present invention further relates to the use of the aqueous compositions according to the invention for the treatment of hair, in particular, for strengthening of hair, for hair color retention, for hair color enhancement, for hair color protection, for shaping of hair, i.e. the curling and straightening of hair, for hair conditioning, for hair smoothening or softening, for hair straightening, for improving manageability of the hair, in particular for improving the combability of the hair.

Aspects of the invention may be further understood with reference to the following non-limiting examples.

### EXAMPLES

### Example 1

### Synthesis of a Gycerol Diglycidyl Ether Based Gallic Acid Derivative

54.1 g 1,3-butandediol, 15 g (0.088 mol COOH) gallic acid and 0.46 g trimethylamine were mixed at room temperature and heated to 70° C. 8.18 g (0.080 mol epoxy groups) glycerol diglycidyl ether

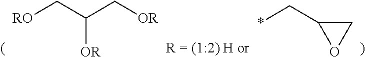

were added dropwise during 30 minutes. The mixture was heated to 90° C. for 12 hrs. The conversion on epoxy groups was 100% (1H-NMR).

A slightly yellowish transparent liquid containing the product essentially consisting of the following structure is obtained

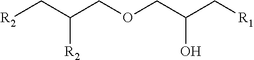

with

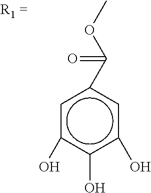

### and R₂ is —OH or

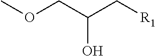

in a ratio 1:1.

### Example 2

### Synthesis of a Propylene Glycol Based Gallic Acid Derivative

56.86 g 1,3-butandediol, 8.95 g (0.0526 mol COOH) gallic acid and 0.28 g trimethylamine were mixed at room temperature and heated to 70° C. 10 g (0.0526 mol epoxy groups) of a glycidyl modified oligopropylene glycol derivative of the structure

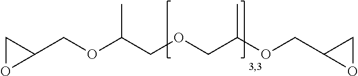

were added dropwise during 1 hr. The mixture was heated to 90° C. for 14 hrs. The conversion on epoxy groups was 100% (1H-NMR).

A yellowish transparent liquid containing the product essentially consisting of the following structure is obtained

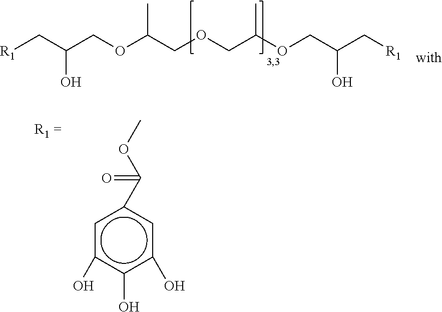

### Applications

### Measurement Methods

### Test Method for Measuring Color and Color Retention of Hair Dyes

The test method for evaluation of the color retention is described in detail in US 2011/0219552 A1. The method determines the change in hair color (Delta E) before and after washing the hair. The color changes were determined by measuring the Hunter L, a and b values on a HunterLab colorimeter before and after washing the color treated hair tress.

The meaning of L, a, b was elaborated in “Practical Modern Hair Science” Trefor Evans and R. Randall Wichett, Alluredbooks, Carol Stream, Ill., 2012. The L value measures the lightness from L=0 (black) to L=100 (white). The color is measured by a from negative value (green) to positive value (red) and b from negative value (blue) to positive value (yellow). For example, a medium blonde has an L, a, b value of L=49, a=12, b=26 and a medium auburn has an L, a, b value of L=26, a=13, b=12.

Delta E was calculated using the following equation to evaluate color change before and after washes.

Delta E_{w}=((Lₜ−L₀)²+(aₜ−a₀)²+(bₜ−b₀)²)^{1/2}

Where L₀, a₀, b₀, (initial color parameters) and Lₜ, aₜ, bₜ (color parameters after washing) are measured Hunter L, a, b color parameters. The larger value of Delta E the greater change of color, so smaller Delta E is desired because it indicates less color loss after washing. Similarly, color enhancement was calculated using the following equation to evaluate initial color depth increase with treatment.

Delta Eₑ=((L₂−L₁)²+(a₂−a₁)²+(b₂−b₁)²)^{1/2}

Where L₂, a₂, b₂, (color parameters for treated colored hair) and L₁, a₁, b₁ (color parameters for untreated colored hair) are measured without washing. Here a larger Delta E is desired because it means more initial color enhancement.

### Example 3: Creamy Shampoo

| S.No | INGREDIENTS | % w/w |
|---|---|---|
| Phase A | | |
| 1 | De-mineralized Water | 21.75 |
| 2 | Disodium EDTA | 0.05 |
| 3 | Carbopol ® Aqua SF-1 Polymer | 6.5 |
| 4 | Galaxy LES | 45 |
| Phase B | | |
| 5 | Triethanolamine | 2 |
| Phase C | | |
| 6 | Galaxy CAPB | 5 |
| 7 | Monemide TM CM | 1 |
| Phase D | | |
| 8 | Ginol 16 | 0.5 |
| 9 | Galaxy 610 | 1.5 |
| Phase E | | |
| 10 | Demineralized water | 10 |
| 11 | Jaguar C13S | 0.1 |
| Phase F | | |
| 12 | Merquat^{TM} 100 | 0.5 |
| | Polymer | |
| 13 | Euxyl ® PE 9010 | 0.4 |
| 14 | Sodium Chloride | 0.7 |
| Phase G | | |
| 15 | gallic acid derivative of example | 5 |
| | 2 | |

### Procedure

1. In the main vessel prepare phase A: Dissolve Disodium EDTA in deionized water, add Carbopol Aqua SF-1 polymer and mix gently, add Galaxy LES and mix gently,
2. Neutralize the phase A with phase B up to pH 6.6 to 7.2
3. Add phase C in to phase AB and mix well
4. Heat the batch up to 80° C.,
5. Add phase D ingredients, and mix well at 80° C. until uniform dispersion.
6. Cool the batch up to 45° C. under stirring.
7. Premix phase E in a separate vessel (use warm water for the dispersion)
8. Add phase E to phase ABCD and mix well.
9. Add phase F ingredients as per given order, mix well after each addition.
10. Finally add phase G mix well & store in a container.

### Application of Creamy Shampoo on the Dyed Hairs

Single bleached platinum hair tresses from International hair importers were dyed using Garnier Red 6.6. Afterwards, washed and dried hair tresses were divided into two-halfs. One half was washed with the creamy shampoo containing the material according to example 2 whereas the other half was washed with the control shampoo (without the inventive additive).

After drying, hair tresses ΔE values were recorded.

The above process was repeated six times.

| | ΔE creamy | ΔE Control |
|---|---|---|
| | shampoo | shampoo |
| No. of | containing | without the |
| wash | the compound | compound of |
| cycles | of example 2 | the invention |
| 1 | 1.25 | 3.74 |
| 2 | 1.7 | 2.41 |
| 3 | 2.41 | 3.14 |
| 4 | 4.03 | 3.8 |
| 5 | 4.9 | 5.45 |
| 6 | 5.22 | 7.45 |

### Example 4: Creamy Shampoo

The experimental protocol outlined under example 3 was repeated using the inventive compound of example 1.

The following results (ΔE) were obtained:

| | ΔE creamy | ΔE Control |
|---|---|---|
| | shampoo containing | shampoo |
| No. of | the inventive | without the |
| wash | material according | inventive |
| cycles | to example 1 | additive |
| 1 | 0.8 | 0.4 |
| 2 | 1.97 | 2.18 |
| 3 | 1.83 | 1.98 |
| 40 | 3.71 | 4 |
| 5 | 3.89 | 4.09 |
| 6 | 3.86 | 4.49 |

The lower ΔE values for the shampoos containing the compounds of the invention according to examples 1 and 2 after six washing cycles mean that less color was lost during the washing operations. The inventive ingredients improve the retention of the artificial hair color. Additionally, it was observed that the tone of the red color on the hair tress half treated with the inventive materials was deeper and the color more homogeneously distributed compared to the second half treated with the control shampoo.

1. A method for the treatment of fibrous amino acid based substrates, preferably hair, which comprises the step of applying a compound of the formula:
R²(—F)₂₋₁₈
wherein
R² is selected from divalent to octadecavalent, optionally substituted, preferably aliphatic hydrocarbon radicals which have up to 100 carbon atoms, and may contain optionally one or more groups selected
from —O—, —NH—, —C(O)—, —C(S)—, tertiary amino groups 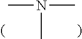
and quaternary ammonium groups 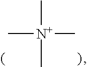
and
F is selected from:
—O—C(O)—R³—R⁴, and
—NR¹—C(O)—R³—r⁴,
the groups F bind to a carbon atom of R²,
wherein
R¹ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 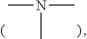
and quaternary ammonium groups 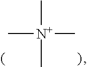
R³ is selected from a single bond or optionally substituted straight-chain, cyclic or branched, saturated, unsaturated or aromatic hydrocarbon radicals which have up to 100 carbon atoms, which optionally contain one or more groups selected from —O—, —NH—, —C(O)—, —C(S)—, tertiary amino groups 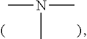
and quaternary ammonium groups 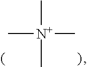
preferably R³ is a single bond, or an ethenylene group, and
R⁴ is selected from the group consisting of di- and trihydroxy-substituted aromatic groups,
wherein the compound excludes tannins,
to said fibrous amino acid based substrates, preferably hair.

2. A method according to claim 1, wherein R⁴ is a group of the formula: 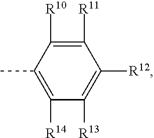
wherein R¹⁰, R¹¹, R¹², R¹³_{, R}¹⁴ are independently selected from R¹, preferably R¹ is hydrogen, or hydroxyl, with the proviso that 2 to 3 groups of R¹⁰ to R¹⁴ are hydroxyl, and wherein the dotted line represents the bond to R³, or is R³ in case R³ is a single bond, or R⁴ is selected from the group consisting of di- and trihydroxy-substituted naphthyl groups.

3. A method according to claim 2, wherein R⁴ is selected from the group consisting of dihydroxyphenyl groups, such as 2,3-dihydroxyphenyl, 2,4-dihydroxyphenyl, 2,5-dihydroxyphenyl, 2,6-dihydroxyphenyl, 3,4-dihydroxyphenyl, and 3,5-dihydroxyphenyl, trihydroxyphenyl groups, such as 2,3,4-trihydroxyphenyl, 2,3,5-trihydroxyphenyl, 2,3,6-trihydroxyphenyl, 2,4,-trihydroxyphenyl, 2,4,6-trihydroxyphenyl, and 3,4,5-trihydroxyphenyl, dihydroxynaphthyl groups, such as 6,7-dihydroxynaphthyl, 5,6-dihydroxynaphthyl, 1,7-dihydroxynaphthyl, 3,7-dihydroxynaphthyl, 1,3-dihydroxynaphthyl, and 1,4-dihydroxynaphthyl, and trihydroxynaphthyl groups, such as 4,6,7-trihydroxynaphthyl, and 1,5,6-trihydroxynaphthyl.

4. Use A method according to claim 1, wherein R⁴ is derived from polyhydroxy aromatic carboxylic acids such as polyhydroxy benzoic acids, such as dihydroxy benzoic acids, e.g. 2,3-dihydroxy benzoic acid, 2,4-dihydroxy benzoic acid, 2,5-dihydroxy benzoic acid, 2,6-dihydroxy benzoic acid, 3,4-dihydroxy benzoic acid, and 3,5-dihydroxy benzoic acid, or trihydroxy benzoic acids, e.g. 2,3,4-trihydroxy benzoic acid, 2,3,5-trihydroxy benzoic acid, 2,3,6-trihydroxy benzoic acid, 2,4,5-trihydroxy benzoic acid, 2,4,6-trihydroxy benzoic acid, and 3,4,5-trihydroxy benzoic acid, polyhydroxy cinnamic acids, such as dihydroxy cinnamic acids, e.g. 3,4-dihydroxy cinnamic acid, or trihydroxy cinnamic acids, e.g. 3,4,5-dihydroxy cinnamic acid, polyhydroxynaphthalene carboxylic acids, such as dihydroxynaphthalene carboxylic acids, e.g. 6,7-dihydroxynaphthalene-2-carboxylic acid, 5,6-dihydroxynaphthalene-2-carboxylic acid, 1,7-dihydroxynaphthalene-2-carboxylic acid, 3,7-dihydroxynaphthalene-2-carboxylic acid, 3-dihydroxynaphthalene-2-carboxylic acid, and 1,4-dihydroxynaphthalene-2-carboxylic acid, or trihydroxynaphthalene carboxylic acids, e.g. 4,6,7-trihydroxynaphthalene-2-carboxylic acid, and 1,5,6-trihydroxynaphthalene-2-carboxylic acid.

5. A method according to claim 1, wherein the optional substituents of the groups R¹, R² and R³ are selected from the groups consisting of hydroxyl, amino and halogen, preferably hydroxyl and amino, and the number of the substituents may be up to 5, preferably 1 to 4.

6. A method according to claim 1, wherein R¹ is selected from the group consisting of hydrogen, n-, iso-, or tert.-C₁-C₂₂-alkyl, C₂-C₂₂-alkoxyalkyl, C₅-C₃₀-cycloalkyl, C₆-C₃₀-aryl, C₆-C₃₀-aryl(C₁-C₆)alkyl, C₆-C₃₀-alkylaryl, C₂-C₂₂-alkenyl, C₂-C₂₂-alkenyloxyalkyl, which optionally can he each substituted by hydroxyl and halogen, and which optionally can contain one or more ether groups (—O—), preferably hydrogen or n-, iso-, or tert.-C₁-C₂₂-alkyl.

7. A method according to claim 1, wherein R² is selected from divalent to decavalent, more preferred divalent to hexavalent, even more preferred divalent, preferably aliphatic, hydrocarbon radicals which have 2 to 30 carbon atoms, more preferred 2 to 20 carbon atoms, even more preferred 2 to 15 carbon atoms and may contain optionally one or more groups selected from —O—, —NH—, —C(O)—, —C(S)—, and wherein R² may optionally be substituted by one or more hydroxyl groups, and preferably R² contains one more groups —O—, and is substituted by one or more hydroxyl groups.

8. A method according to an claim 1, wherein R² is selected from R²′ being selected from the group consisting of:
divalent to octadecavalent, preferably divalent to hexavalent hydrocarbyl groups, more preferably divalent hydrocarbyl groups, derived from aliphatic polyols having more than three carbon atoms such as alkane di-, tri- and tetraols, e.g. 1,6 hexandiol, trimethylolpropane, and pentaerythritol,
divalent to octadecavalent, preferably divalent to hexavalent hydrocarbyl groups, more preferably divalent hydrocarbyl groups, comprising at least one ether group, and optionally having one or more hydroxyl substituents, derived from polyalkyleneoxides, such as ethylene oxide-, propylene oxide- and/or butylene oxide-based polyethers, e.g. derived from polyethylene glycols, like diethylene glycol, triethylene glycol, tetraethylene glycol, and pentaethylene glycol etc., or derived from polypropylene glycols, like dipropylene glycol (e.g, derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, etc., derived from mixed ethylene oxide and butylene oxide based copolyethers, derived from mixed propylene oxide and butylene oxide based copolyethers, and derived from mixed ethylene oxide and propylene oxide and butylene oxide based copolyethers,
divalent to octadecavalent, preferably divalent to hexavalent hydrocarbyl groups, more preferably divalent hydrocarbyl groups, optionally comprising at least one ether group, and optionally having one or more hydroxyl substituents, derived from oligoglycerols, such as diglycerol, triglycerol, tetraglycerol, pentaglycerol, hexaglycerol, and the statistically distributed oligomeric condensation products of glycerol, such as
divalent to octadecavalent, preferably divalent to hexavalent hydrocarbyl groups, more preferably divalent hydrocarbyl groups, derived from compounds comprising at least one glycidoxy group, such as diglycidyl ether, glycerol diglycidyl ether, and glycerol triglycidylether,
divalent to octadecavalent, preferably divalent to hexavalent hydrocarbyl groups, more preferably divalent hydrocarbyl groups, comprising at least one ether group, and optionally having one or more hydroxyl substituents, derived from polyol alkylene oxide addition products, e.g. the addition products of ethylene oxide and/or propylene oxide to polyols such as ethylene glycol, 1,2 propylene glycol, glycerol, trimethylolpropane, pentaerythritol, sorbitol and sucrose,
divalent to octadecavalent, preferably divalent to hexavalent hydrocarbyl groups, more preferably divalent hydrocarbyl groups, comprising at least one ether group, and optionally having one or more hydroxyl and/or amino substituents, derived from polyamine alkylene oxide addition products, e.g. the addition products of ethylene oxide and/or propylene oxide to ethylenediamine, diethylene triamine, or derived from alkylene oxide addition products of ethanol amine,
divalent to octadecavalent, preferably divalent to hexavalent hydrocarbyl groups, more preferably divalent hydrocarbyl groups, comprising at least one ester group, and having one or more hydroxyl substituents, e.g. derived from polyesters, preferably derived from the condensation of di- to hexavalent carboxylic acids, e.g. maleic acid, succinic acid, adipic acid, sebacic acid, itaconic acid, tartaric acid, trimellitic acid with alkylene oxides, such as ethylene oxide, propylene oxide, butylene oxide, and compounds comprising at least one glycidoxy group, such as glycidol, diglycidyl ether, glycerol diglycidyl ether, glycerol triglycidyl ether, in particular the condensation products of succinic acid, maleic acid and tartaric acid to glycerol diglycidyl ether,
further R²′ presents polyalkylene oxy groups, preferably of the general formula:
—[CH₂CH₂O]_{q1}—[CH₂CH(CH₃)O]ᵣ₁—[CH₂CH(C₂H₅)O]ₛ₁—{[CH₂CH₂]_{q2}—[CH₂CH(CH₃)]ᵣ₂—[CH₂CH(C₂H₅)]ₛ₂}—
with
q1=0 to 49, preferred 0 to 10, more preferred 1 to 10, even more preferred 1 to 5,
r1=0 to 32, preferred 0 to 10, more preferred 1 to 10, even more preferred 1 to 5,
s1=0 to 24, preferred 0 to 10, more preferred 1 to 10, even more preferred 1 to 5,
q2=0 or 1,
r2=0 or 1,
s2=0 or 1, and
Σ(q2+r4+s2)=1,
with the proviso that the sum of the carbon atoms in such polyalkylene oxide groups is 2 to 100, preferred 2 to 50, more preferred 2 to 30, even more preferred 2 to 20, specific 2 to 15,
divalent hydrocarbyl groups, derived from oligoglycerols of the general formula:
—[CH₂CH(R⁷)CH₂O]ₜ₁—[CH₂CH(R⁷)CH₂)]ₜ₂—
with
t1=0 to 32, preferred 0 to 10, more preferred 1 to 10, even more preferred 1 to 5, specifically 1 and 2,
t2=1,
R⁷=OH or F, wherein F is as defined above, preferably -—O—C(O)—R³—C(O)OH,
with the proviso that the sum of the carbon atoms is 2 to 100, preferred 2 to 50, more preferred 2 to 30, even more preferred 2 to 20, specific 2 to 15, and
divalent hydrocarbyl groups, comprising at least one ester group of the general formulae
—[CH₂CH₂O]_{q1}—R⁸—[CH₂O]_{q1}—[CH₂CH₂]_{q2}—
with q1 can be the same or different and are as defined above and q2=1
and
—[CH₂CH(R⁷)CH₂O]ₜ₁—R⁸—[CH₂CH(R⁷)CH₂O]ₜ₁—[CH₂CH(R⁷)CH₂)]ₜ₂—
with t1, t2 and R⁷ as defined above and
R⁸ being selected from —C(O)C(O)O—, —C(O)(CH₂)₁₋₈C(O)O—, such as being derived from succinic acid, adipic acid, sebacic acid, or —C(O)(C₆H₄)C(O)O—, i.e. derived from phthalic and terephthalic acid, —C(O)CH═CHC(O)O—, —C(O)C(═CH₂)≤CH₂C(O)O—, —C(O)CH(OH)CH(OH)C(O)O—,
with the proviso that the sum of the carbon atoms is 2 to 100, preferred 2 to 50, more preferred 2 to 30, even more preferred 2 to 20, specifically 2 to 15.

9. (canceled)

10. A method according to claim 1, wherein the compound is selected from the group consisting of:
a compound of the formula: 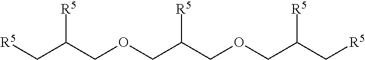
wherein R⁵ is selected from the group consisting of hydroxy or F, wherein F is as defined above, with the proviso that at least two of R⁵ are F; a compound of the formula: 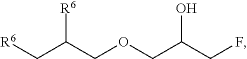
wherein one of R⁶ is hydroxy and one of R⁶ is a group of the formula 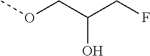
wherein F is as defined above and the dotted line is the bond to the carbon atom:
a compound which is a mixture of the following two isomers; 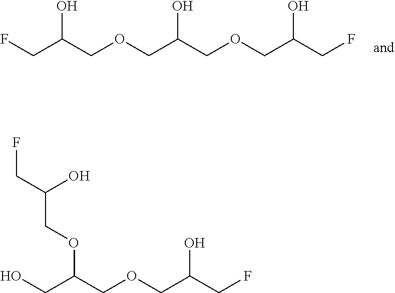
wherein F is as defined above; and
a compound of the formula: 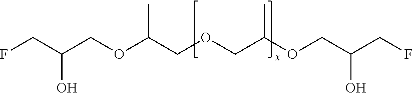
wherein x is from 1 to 10, preferably 1 to 5, and F is as defined above.

11.-13. (canceled)

14. A method according to claim 1, wherein R³ is selected from a single bond and straight-chain, cyclic or branched, saturated, unsaturated or aromatic hydrocarbon radicals which have up to 18, preferably up to 12, more preferably up to 10 carbon atoms, and which optionally contain one or more groups selected from —O—, —NH—, —C(O)—, and wherein R³ is optionally substituted by one or more groups selected from hydroxyl groups, amino groups, and carboxy groups, preferably R³ is single bond or ethenylene.

15. A method according to claim 1, wherein R² is an aliphatic hydrocarbyl group which is not derived from a hexose; or
R² is not derived from a carbohydrate, or a sugar alcohol; or
the compound excludes carbohydrate- or sugar alcohol esters of gallic acid.

16.-17. (canceled)

18. A compound of the formula:
R²″(—F)₂₋₁₈
wherein R²″ is selected from divalent to octadecavalent, optionally substituted, aliphatic hydrocarbon radicals which have up to 100 carbon atoms, and may contain optionally one or more groups selected. from —O—, —NH—, —C(O)—, —C(S)—, tertiary amino groups 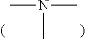
and quaternary ammonium groups 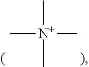
and
F is selected from selected from:
—O—C(O)—R³—R⁴, and
—NR¹—CO(O)—R³—R⁴,
the groups F bind to a carbon atom of R²″, wherein R²″ is not derived from a carbohydrate (saccharide), or a sugar alcohol, such as ethylene glycol or glycerol, and
wherein
R¹ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 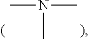
and quaternary ammonium groups 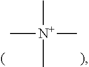
R³ is selected from a single bond or optionally substituted straight-chain, cyclic or branched, saturated, unsaturated or aromatic hydrocarbon radicals which have up to 100 carbon atoms, which optionally contain one or more groups selected from —O—, —NH—, —C(O)—, —C(S)—, tertiary amino groups 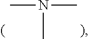
and quaternary ammonium groups 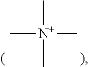
preferably R³ is a single bond, or an ethenylene group, and
R⁴ is selected from the group consisting of di- and trihydroxy-substituted aromatic groups.

19. A compound according to claim 18, wherein the compound excludes tannins.

20. A compound according to any of the claim 18, which has the formula:
R²′(—F)₂₋₁₈
wherein
R²′ and F are as defined above.

21. A compound according to claim 18, which has the formula: 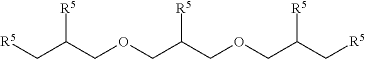
wherein R⁵ is selected from the group consisting of hydroxy or F, wherein F is as defined above, with the proviso that at least two of R⁵ are F.

22. A compound according to claim 18, wherein the compound has the formula: 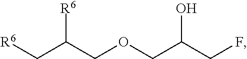
wherein one of R⁶ is hydroxy and one of R⁵ is a group of the formula 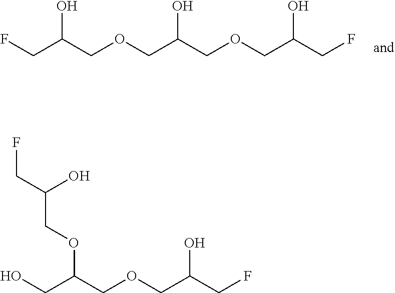
wherein F is as defined above and the dotted line is the bond to the carbon atom.

23. A compound according to claim 18, wherein the compound is a mixture of the following two isomers: 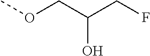
wherein F is as defined above.

24. A compound according to claim 18, wherein the compound has the formula: 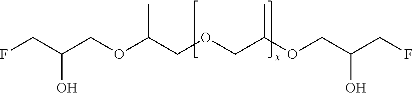
herein x is from 1 to 10, preferably 1 to 5, and F is as defined above.

25. An aqueous composition comprising the compound according to claim 18.

26. An aqueous composition comprising the compound as defined in claim 1, and at least one further component selected from the group consisting of surfactants, and metal salts, preferably Zn²⁺, Fe²⁺ and/or Fe³⁺ salts.

27. (canceled)

28. The aqueous composition according to claim 26, wherein the weight ratio of said surfactant and/or metal salt to said inventive compound is at least 0.06, more preferred 0.06 to 5, more preferred 0.06 to 3, even more preferred 0.1 to 3, specifically 0.1 to 1.

29. The aqueous co -position according to claim 25, comprising 0.05 to 30%, preferred 0.5 to 30%, more preferred 1 to 30%, even more preferred 1 to 20%, specifically 1 to 10 of said compound, wherein each percentage is per weight based on the total weight of the aqueous composition.

30.-32. (canceled)

33. The aqueous composition according to claim 25, comprising at least one additional additive, selected front the group consisting of
a) organic diluents or solvents,
b) proteins, preferably keratin,
c) emollients or fatty substances,
d) preservatives,
e) skin protecting ingredients,
f) conditioning agents,
g) oxidizing agents,
h) reducing agents,
i) tannins,
j) metal salts,
k) further auxiliaries selected from pH adjusting agents, thickeners, lipids, amino acids, sugars, fragrances, sunscreen agents, vitamins, pearlescent agents, gelling agents, trace elements, sequestering agents, antioxidants, humectants, anti-hair loss agents, anti-dandruff agents, propellants, ceramides, polymers, in particular film-forming polymers, fillers, nacres, colorants, in particular pigments and dyes, and mixtures thereof,
with the proviso that oxidizing agents and reducing agents are not present simultaneously in a given composition.

34. The aqueous composition according to claim 25, having the composition:

| Ingredient | Weight- % |
|---|---|
| the compound as defined in claim 25 | 0.05 to 30, preferably 1 to 10 |
| hydrocarbon or silicone based surfactant | 0 to 15, preferably 0.05 to 5 |
| water | q.s. to add to 100 |
| diluents/solvents | 0 to 95, preferably 0.1 to 95, preferred 10 to |
| | 95, more preferred 20 to 95, even more |
| | preferred 20 to 50 and 50 to 95 |
| protein, preferred keratin | 0 to 15, preferably 0 to 10, such as 0.01 to 5 |
| emollients/fatty substance | 0 to 15, preferably 0 to 10, more preferred 0 to |
| | 5, such as 0.01 to 5 |
| preservatives | 0 to 5, preferably 0 to 3, more preferred 0 to 2, |
| | such as 0.01 to 2.5 |
| skin protecting ingredients | 0 to 15, preferred 0 to 10, more preferred 0 to |
| | 5, even more preferred 0 to 1, specifically 0 to |
| | 0.1, e.g. 0.01 to 5 |
| conditioning agents | 0 to 15, preferred 0 to 10, more preferred 0 to |
| | 5, even more preferred 0 to 1, specifically 0 to |
| | 0.1, e.g. 0.01 to 5 |
| oxidizing agents | 0 to 15, preferred 0 to 10, more preferred 0 to |
| | 5, even more preferred 0 to 2, e.g. 0.01 to 5 |
| reducing agents | 0 to 15, preferred 0 to 10, more preferred 0 to |
| | 5, even more preferred 0 to 2, e.g. 0.01 to 5 |
| tannins | 0 to 15, preferred 0 to 10, more preferred 0 to |
| | 5, even more preferred 0 to 2, e.g. 0.01 to 5 |
| metal salts, such iron or zinc salts, | 0 to 15, preferred 0 to 10, more preferred 0 to |
| | 5, even more preferred 0 to 2, e.g. 0.01 to 5 |
| hair dyeing agent | 0 to 15, preferred 0 to 10, more preferred 0 to |
| | 5, even more preferred 0 to 2, e.g. 0.01 to 5 |
| further auxiliary agents | 0 to 15, preferred 0 to 10, more preferred 0 to |
| | 5, even more preferred 0 to 2, e.g. 0.01 to 5 |

wherein the wt-percentages relate to the complete weight of the aqueous composition.

35. A cosmetic composition comprising at least one compound according to claim 1.

36. A cosmetic composition according to claim 35, which is for use in hair treatment, preferably for use in hair coloring treatment.

37. A cosmetic composition according to claim 36, which is selected from the group consisting of a hair shampoo composition, hair care composition, hair conditioning composition, hair strengthening composition, hair coloration or dyeing composition, hair combability improving composition, anti-fizz composition, hair rinse-off and leave-on compositions.

38. A method for the treatment of hair which comprises the steps of providing a cosmetic composition according to claim 25, and applying said cosmetic composition to said hair.

39. A method for the treatment of hair according to claim 38, which further comprises the step of dyeing the hair.

40. A method for the treatment of hair, which comprises the steps of
contacting the hair with the cosmetic composition according to claim 35,
optionally contacting the hair with a composition containing a metal salt of Fe and/or Zn, and
drying the hair.

41.-42. (canceled)

43. A cosmetic composition comprising at least one compound according to claim 18.

44. A cosmetic composition comprising at least one aqueous composition according to claim 25.

45. A method for the treatment of hair, which comprises the steps of
contacting the hair with the cosmetic composition according to claim 43,
optionally contacting the hair with a composition containing a metal salt of Fe and/or Zn, and
drying the hair.
